(19) United States
(12) Patent Application Publication
(10) Pub. No.: US 2024/0168041 A1
(43) Pub. Date: May 23, 2024

(54) SYSTEMS AND METHODS ENABLING PHARMACOLOGY SAMPLE TESTING OF PATCH-CLAMP SAMPLES

(71) Applicants: Georgia Tech Research Corporation, Atlanta, GA (US); Emory University, Atlanta, GA (US)

(72) Inventors: Riley E. Perszyk, Atlanta, GA (US); Craig Richard Forest, Atlanta, GA (US); Andrew Jenkins, Atlanta, GA (US); Stephen Traynelis, Atlanta, GA (US); Mighten C. Yip, Atlanta, GA (US)

(21) Appl. No.: 18/282,989

(22) PCT Filed: Apr. 14, 2022
(86) PCT No.: PCT/US2022/024743

§ 371 Date: Sep. 19, 2023

Related U.S. Application Data
(60) Provisional application No. 63/174,846, filed on Apr. 14, 2021.

(51) Int. Cl. G01N 35/00; G01N 33/487

(52) U.S. Cl. CPC G01N 35/00584; G01N 33/48728

(57) ABSTRACT

An exemplary embodiment of the present disclosure provides a system comprising a pipette tip manipulable between a sample area and an active agent exposure zone and a controller configured to manipulate the pipette tip between the sample area and the active agent exposure zone, wherein when the pipette tip comprises at least a portion of a biological sample target, the controller is further configured to isolate at least a portion of the biological sample target from one or more biological sample targets. Also disclosed is a method for detecting an effect of an active agent on a cell, by forming a high-resistance seal between a cell and an apertured surface, isolating at least a portion of the cell, translocating the apertured surface in one or more translations within an x-plane, a y-plane, a z-plane, or combinations thereof, and exposing the portion of the cell to an active agent.

### CROSS-REFERENCE TO RELATED APPLICATIONS

This application claims the benefit of U.S. Provisional Application Ser. No. 63/174,846, filed on 14 Apr. 2021, which is incorporated herein by reference in its entirety as if fully set forth below.

### FEDERALLY SPONSORED RESEARCH STATEMENT

This invention was made with government support under grant/award number 5-R01-NS102727-04, awarded by the National Institutes of Health, grant/award number U01MH106027, awarded by the National Institute of Mental Health, grant/award number R01 NS102727, awarded by the National Institutes of Neurological Disorders on Stroke, grant/award number R01 EY023173, awarded by the National Eye Institute, grant/award number T90 DA032466, awarded by the National Institute of Drug Abuse, and R35 NS111619, awarded by the National Institutes of Neurological Disorders on Stroke. The government has certain rights in the invention.

### FIELD OF THE DISCLOSURE

The various embodiments of the present disclosure relate generally to sample testing systems and methods, and more particularly to biological sample testing systems and methods that enable intracellular pharmacological electrophysiology sampling of a biological sample.

### BACKGROUND

Patch-clamp recording is a gold-standard single-cell electrophysiology technique that has been widely used to discover foundational biophysical properties of excitable cells. In neuroscience, the superior sensitivity and resolution of patch-clamp recording has made it an indispensable tool for discovering the tenants of ion channel activity, synaptic integration, plasticity and network connectivity in a variety of experimental preparations from cultured cells to living tissue. Patch-clamp recording has the ability to accurately measure the time-course of postsynaptic or post-junctional currents and can fully resolve ion flux and the rapid transitions of individual ionotropic receptors.

Many pathological conditions such as migraine, hypertension and hypersomnia arise from dysfunctions in cell membrane-bound ion channels. Patch clamp recording is the highest resolution tool available to measure the responses of these channels to changes in the cell environment, such as from interactions with active agents or drugs. Patch-clamp recording provides an ability to measure extremely small (on the order of 10-12 Amperes) electrical currents arising as a result of single or multiple ion channels trafficking charged ions across the cell membrane. However, this high-resolution technique requires extensive effort and time. Patch-clamp recording involved pressing a glass micropipette (namely the aperture of the micropipette) against a cell membrane to achieve a high-resistance (>1 GQ) seal, or “gigaseal,” with the cell membrane. When the cell is patched, the patch-clamp recording of that particular cell can take place.

Examples of systems and devices configured for autonomous intracellular electrophysiology are disclosed in U.S. Pat. Nos. 10,733,419 and 11,188,737, each of which are incorporated herein by reference in its entirety as if fully set forth below. Robotic vision, pipette pressure control, and electrode cleaning enables the resulting “patcherBot” to execute the basic steps required to perform patch-clamp electrophysiology without human intervention. Utilizing these automated methods allows for the acceleration of electrophysiology experimentation by reducing the process times of many steps as well as drastically decreasing the amount of required operator-rig interfacing time. The patcherBot is capable of patching over 30 cells sequentially, can run unattended for over 4 hours, and operates at about a 70% success rate (reaching the whole-cell patch-clamp configuration per patching attempt). These advances enable the patcherBot to record spontaneous activity or voltage-dependent biological phenomena, and they can be multiplexed within a single preparation to record from multiple cells simultaneously. Thus, the patcherBot is highly proficient at addressing questions such as connectomics or intrinsic properties of neurons. Despite its many capabilities, this technology cannot perform many assays on ligand-gated ionotropic receptors or pharmacological studies.

When studying the effects of drugs on an individual cell, one would need to either isolate and introduce the drug to the isolated cell or introduce the drug to the tissue sample or group of cells, which would limit the ability to study immediate effects on more than one cell at a time. Isolating an individual cell is difficult as the cells are delicate and requires a balance between the force holding the cell membrane against the micropipette and the force to separate a cell from neighboring cells. This is a major barrier to facilitating high-throughput drug screening.

### BRIEF SUMMARY

The present disclosure relates to biological sample testing systems and methods that enable intracellular pharmacological electrophysiology sampling of a biological sample. An exemplary embodiment of the present disclosure provides a system comprising a pipette tip and a controller. The pipette tip can be manipulable between a sample area and an active agent exposure zone. The sample area can include one or more biological sample targets. The controller can be configured to manipulate the pipette tip between the sample area and the active agent exposure zone. When the pipette tip comprises at least a portion of a biological sample target, the controller can be further configured to isolate at least a portion of the biological sample target from the one or more biological sample targets.

In any of the embodiments disclosed herein, the active agent exposure zone can include a delivery port configured to distribute an active agent to the active agent exposure zone.

In any of the embodiments disclosed herein, the system can further include a pressure controller in communication with the pipette tip and configured to control a pressure at the pipette tip.

In any of the embodiments disclosed herein, the controller can further be configured to control the pipette tip to form a high-resistance seal with the portion of the biological sample target such that the portion of the biological sample target becomes sealed to the pipette tip.

In any of the embodiments disclosed herein, the controller can further be configured to translocate the pipette tip and the portion of the biological sample target from the sample area to the active agent exposure zone.

In any of the embodiments disclosed herein, the controller can further be configured to translocate the pipette tip from the sample area with at least one of a pre-determined pattern of motion, a pre-determined speed, and/or a pre-determined pressure.

In any of the embodiments disclosed herein, the pre-determined pattern of motion can include one or more translations within an x-plane, a y-plane, a z-plane, or combinations thereof.

In any of the embodiments disclosed herein, the pre-determined pattern of motion can include one or more of a spiral motion, a helical motion, a wave motion, a square motion, a triangle motion, a sawtooth motion, a reciprocating motion, an oscillating motion, a rotary motion, a linear motion, a translatory motion, a rectilinear motion, a curvilinear motion, a parabolic motion, a circulatory motion, a vibratory motion, a periodic motion, and combinations thereof.

In any of the embodiments disclosed herein, the controller can further be configured to modify at least one of the pre-determined pattern of motion, the pre-determined speed, and/or the pre-determined pressure based on a change in the high-resistance seal between the pipette tip and the portion of the biological sample target.

In any of the embodiments disclosed herein, the controller can further be configured to control the pipette tip into contact with at least a portion of a biological sample target from the biological sample targets to test for at least one characteristic of the biological sample target.

In any of the embodiments disclosed herein, the controller can further be configured to measure a first electrical property of the portion of the biological sample target at the pipette tip.

In any of the embodiments disclosed herein, the controller can further be configured to determine whether the pipette tip has been contacted with at least a portion of the biological sample target by constructing a first temporal series of electrical property measurements.

In any of the embodiments disclosed herein, the first temporal series of electrical property measurements can be indicative of initiating contact between the pipette tip and the portion of the biological sample target to form a high-resistance seal.

In any of the embodiments disclosed herein, the controller can further be configured to control the delivery port and the pipette tip in contact with at least a portion of a biological sample target within the active agent exposure zone to test for at least one characteristic of the biological sample target before, during, and after exposure to the active agent from the delivery port.

In any of the embodiments disclosed herein, the controller can further be configured to control an amount of the active agent distributed from the delivery port.

In any of the embodiments disclosed herein, the controller can further be configured to control an amount of the active agent distributed to at least a portion of the biological sample target sealed to the pipette tip.

In any of the embodiments disclosed herein, the controller can further be configured to measure a second electrical property at the pipette tip indicative of the high-resistance seal of the portion of the biological sample target.

In any of the embodiments disclosed herein, the controller can further be configured to determine whether the pipette tip is contacting with the portion of the biological sample target by constructing a second temporal series of electrical property measurements.

In any of the embodiments disclosed herein, the second temporal series of electrical property measurements can be indicative of an effect of an active agent on the portion of the biological sample target.

In any of the embodiments disclosed herein, the controller can further be configured to determine whether the pipette tip is losing contact with at least a portion of the biological sample target by detecting a change in the high-resistance seal between the pipette tip and the biological sample target from the first and second temporal series of electrical property measurements.

In any of the embodiments disclosed herein, the first electrical property, second electrical property, first temporal series of electrical property measurements, and second temporal series of electrical property measurements can include at least one of a current, a capacitance, a resistivity, a conductivity, a temperature coefficient of resistance, a dielectric strength, thermoelectricity, or a combination thereof.

In any of the embodiments disclosed herein, the controller can further be configured to control the pipette tip between the sample area and a cleaning area, the cleaning area configured to contain cleaning solution and fluidically separate the cleaning solution from the sample area.

In any of the embodiments disclosed herein, the controller can further be configured to automate a whole-cell patch clamp recording of one or more biological sample targets.

In any of the embodiments disclosed herein, the controller can further be configured to automate an outside-out patch clamp recording of one or more biological sample targets.

An exemplary embodiment of the present disclosure provides a method for detecting an effect of an active agent on a cell, the method can include manipulating a pipette tip between a sample area and an active agent exposure zone; controlling the pipette tip through the sample area, the sample area comprising biological sample targets; isolating a biological sample target from the biological sample targets; and controlling the pipette tip through the active agent exposure zone.

In any of the embodiments disclosed herein, controlling the pipette tip through the sample area can include contacting the pipette tip with the biological sample target of the biological sample targets and measuring at least one characteristic of the biological sample target at the pipette tip.

In any of the embodiments disclosed herein, the method can further include one of achieving a whole-cell conformation such that the biological sample target at the pipette tip comprises an entire biological sample target or achieving a patch conformation such that the biological sample target at the pipette tip comprises a portion of the biological sample target.

In any of the embodiments disclosed herein, isolating a biological sample target from the biological sample targets can include forming a gigaseal between the pipette tip and the biological sample target and translocating the pipette tip in one or more translations within an x-plane, a y-plane, a z-plane, or combinations thereof.

In any of the embodiments disclosed herein, isolating a biological sample target from the biological sample targets can further include translocating the pipette tip from the sample area by at least one of a pre-determined pattern of motion, a pre-determined speed, and/or a pre-determined pressure.

In any of the embodiments disclosed herein, the pre-determined pattern of motion can include one or more of a spiral motion, a helical motion, a wave motion, a square motion, a triangle motion, a sawtooth motion, a reciprocating motion, an oscillating motion, a rotary motion, a linear motion, a translatory motion, a rectilinear motion, a curvilinear motion, a parabolic motion, a circulatory motion, a vibratory motion, a periodic motion, and combinations thereof.

In any of the embodiments disclosed herein, the method can further include modifying at least one of the pre-determined pattern of motion, the pre-determined speed, and/or the pre-determined pressure based on a change in one or more electrical properties between the pipette tip and the biological sample target.

In any of the embodiments disclosed herein, controlling the pipette tip through the active agent exposure zone can include contacting the pipette tip and the biological sample target of the biological sample targets with a perfusion of one or more active agents within the active agent exposure zone and measuring at least one characteristic of the biological sample target at the pipette tip. At least one characteristic of the biological sample target can be selected from the group consisting of an electrical activity, a molecular activity, a drug screening property, biological sample target type, a biophysical property, and a genetic property.

In any of the embodiments disclosed herein, the method can further include automating the steps of the method via one or more output signals from a controller executing commands in response to one or more input signals. The controller can execute instructions stored in a memory that, when executed by the controller, control the controller to execute the commands.

An exemplary embodiment of the present disclosure provides a method for detecting an effect of an active agent on a cell, the method can include forming a high-resistance seal between a cell within a sample of cells and an apertured surface; isolating at least a portion of a cell from the sample of cells, wherein isolating the cell comprises translocating the apertured surface from the sample of cells in one or more translations within an x-plane, a y-plane, a z-plane, or combinations thereof; and exposing the cell to an active agent.

In any of the embodiments disclosed herein, the method can further include one of achieving a whole-cell conformation such that the cell at the pipette tip comprises an entire cell or achieving a patch conformation such that the cell at the pipette tip comprises a cell membrane patch.

In any of the embodiments disclosed herein, isolating the cell or the cell membrane patch from the sample of cells can further include translocating the apertured surface from the sample of cells in a pre-determined pattern of motion.

In any of the embodiments disclosed herein, the pre-determined pattern of motion can include one or more of a spiral motion, a helical motion, a wave motion, a square motion, a triangle motion, a sawtooth motion, a reciprocating motion, an oscillating motion, a rotary motion, a linear motion, a translatory motion, a rectilinear motion, a curvilinear motion, a parabolic motion, a circulatory motion, a vibratory motion, a periodic motion, and combinations thereof.

In any of the embodiments disclosed herein, the method can further include measuring a first resistance at the apertured surface. The first resistance measurement can be indicative of forming the high-resistance seal between the apertured surface and the cell. The method can further include measuring a second resistance at the apertured surface. The second resistance measurement can be indicative of maintaining the high-resistance seal between the apertured surface and the cell as the apertured surface is translocated between a sample area and an active agent exposure zone.

In any of the embodiments disclosed herein, the cell to an active agent can include testing for at least one characteristic of the cell.

In any of the embodiments disclosed herein, at least one characteristic of the cell can be selected from the group consisting of an electrical activity, a molecular activity, a drug screening property, biological sample target type, a biophysical property, and a genetic property.

In any of the embodiments disclosed herein, forming a high-resistance seal between a cell within a sample of cells and an apertured surface further can include measuring a first temporal series of resistance measurements.

In any of the embodiments disclosed herein, isolating the cell from the sample of cells further can include measuring a second temporal series of resistance measurements.

In any of the embodiments disclosed herein, measuring a change in the first and second temporal series of resistance measurements can be indicative of a change in the high-resistance seal between the apertured surface and the cell during isolating the cell from the sample of cells.

In any of the embodiments disclosed herein, the method can further include automating the steps of the method via one or more output signals from a controller executing commands in response to one or more input signals. The controller can execute instructions stored in a memory that, when executed by the controller, control the controller to execute the commands.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A is a schematic, pictorial illustration of a system having a pipette tip and a controller, in accordance with an exemplary embodiment of the present disclosure.

FIGS. 1B and 1C are schematic, pictorial illustrations of a system isolating a cell and exposing the cell to an active agent, in accordance with an exemplary embodiment of the present disclosure.

FIGS. 1D and 1E are schematic, pictorial illustrations of a prior implementation of a patch-clamp system (FIG. 1D) and a system for isolating a cell and exposing the isolated cell to an active agent (FIG. 1E), in accordance with an exemplary embodiment of the present disclosure.

FIG. 2A provides an image of a system having a pipette tip and a controller, in accordance with an exemplary embodiment of the present disclosure.

FIG. 2B is a schematic, pictorial illustration of the distance a pipette tip may translate during experimentation, in accordance with an exemplary embodiment of the present disclosure.

FIG. 2C is a current recording versus time testing the ability of a pipette tip to return to a critical location, and the corresponding exchange time, required for sequential patching of a cell or sample of cells, in accordance with an exemplary embodiment of the present disclosure.

FIGS. 2D through 2F provide an example cell lifting procedure, in accordance with an exemplary embodiment of the present disclosure. FIG. 2D provides an image of an isolated cell in the whole-cell conformation before lifting; FIG. 2E provides an example spiral motion path employed to lift isolated cells; and FIG. 2F provides a resistance plot showing a high-resistance seal is robustly maintained during the lifting process.

FIGS. 2G through 2I provide an example patch pulling procedure, in accordance with an exemplary embodiment of the present disclosure. FIG. 2G provides an image of a cell in the whole-cell conformation before pulling an outside-out patch; FIG. 2H provides an example arc path employed to pull outside-out patches; and FIG. 2I provides capacitance and resistance plots showing successful high-resistance, low-capacitance outside-out patches.

FIGS. 3A and 3B provide exemplary fast-solution exchange electrophysiological experimental results using the system, in accordance with an exemplary embodiment of the present disclosure.

FIG. 4 provides a representative experimental timeline of the system operation with recording results and plots of capacitance and resistance for each electrophysiological experiment conducted during the timeline, in accordance with an exemplary embodiment of the present disclosure.

FIGS. 5A through 5F provides various steps to achieve an example case study analyzing the time-constant concentration response of deactivation of a cell receptor exposed to a drug using the system, in accordance with an exemplary embodiment of the present disclosure.

FIGS. 6A through 6F are schematic, pictorial illustrations of example solution exchange protocols programmed into the system, in accordance with an exemplary embodiment of the present disclosure.

FIGS. 7A through 7G provide an image of a pipette contacting a cell and example resulting voltage-clamp protocol and current-clamp protocol examples that can be accomplished by and/or programmed into the system, in accordance with an exemplary embodiment of the present disclosure.

FIG. 8 is a flowchart illustrating an example method for detecting an effect of an active agent on a cell using the system, in accordance with an exemplary embodiment of the present disclosure.

FIG. 9 is a flowchart illustrating another example method for detecting an effect of an active agent on a cell using the system, in accordance with an exemplary embodiment of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

Patch-clamp recording involves pressing a glass micropipette (namely the aperture) against a cell membrane to achieve a high-resistance (>1 GQ) seal, or “gigaseal”, with the cell membrane. When the glass pipette tip contacts a cell membrane, either under ambient pressure or a light vacuum suction, the cell membrane will bind with the glass pipette tip such that an electrical seal preventing ions, such as cations, from leaking out between the cell membrane and the glass pipette tip. Generally, the light suction on the cell membrane generates the high-resistance seal. These gigaseals only form when using clean micropipettes, necessitating an operator to replace the contaminated pipette with a fresh one between every contact with a cell membrane. This is a major barrier to facilitating high-throughput drug screening. Whole-cell patch clamp electrophysiology of neurons enables the recording of electrical events in cells with great precision and supports a wide diversity of morphological and molecular analysis experiments important for the understanding of single-cell and network functions in a cell when exposed to an active agent.

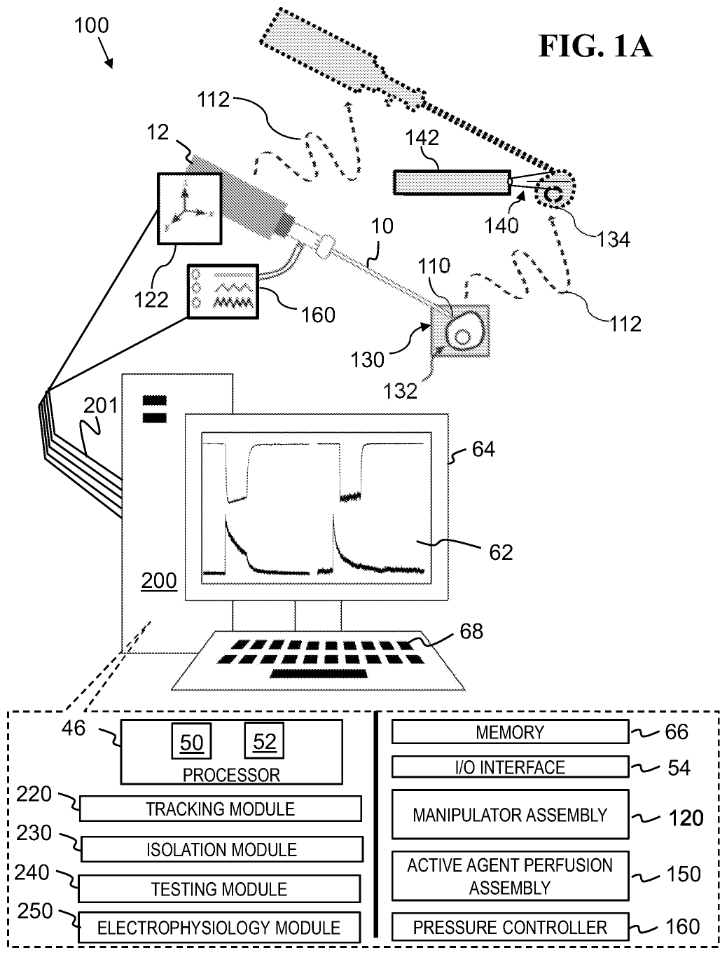

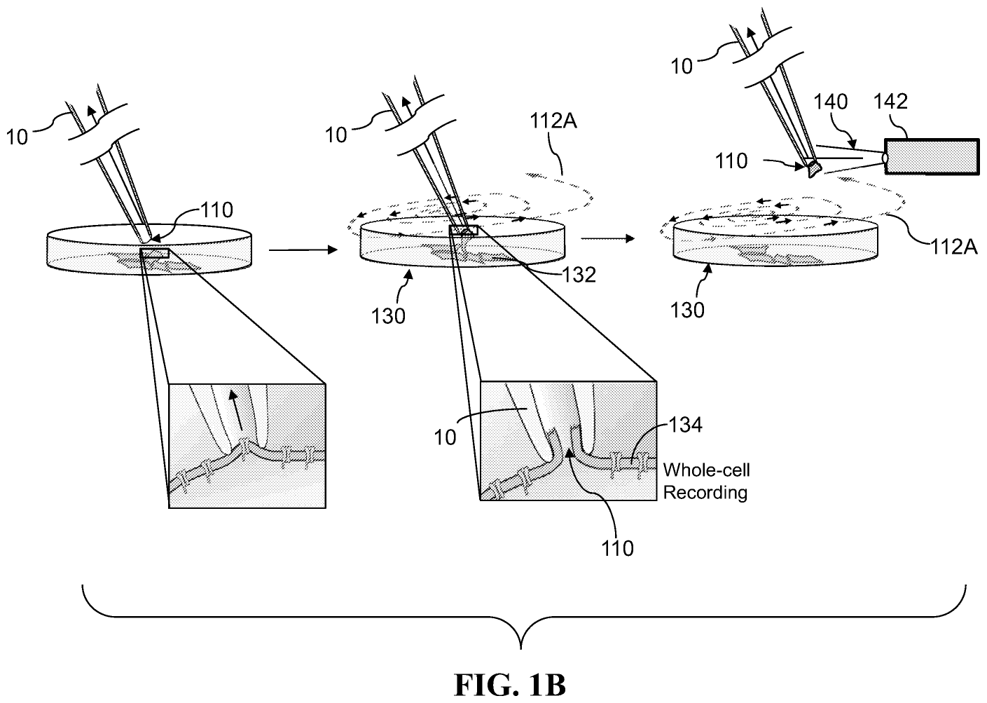

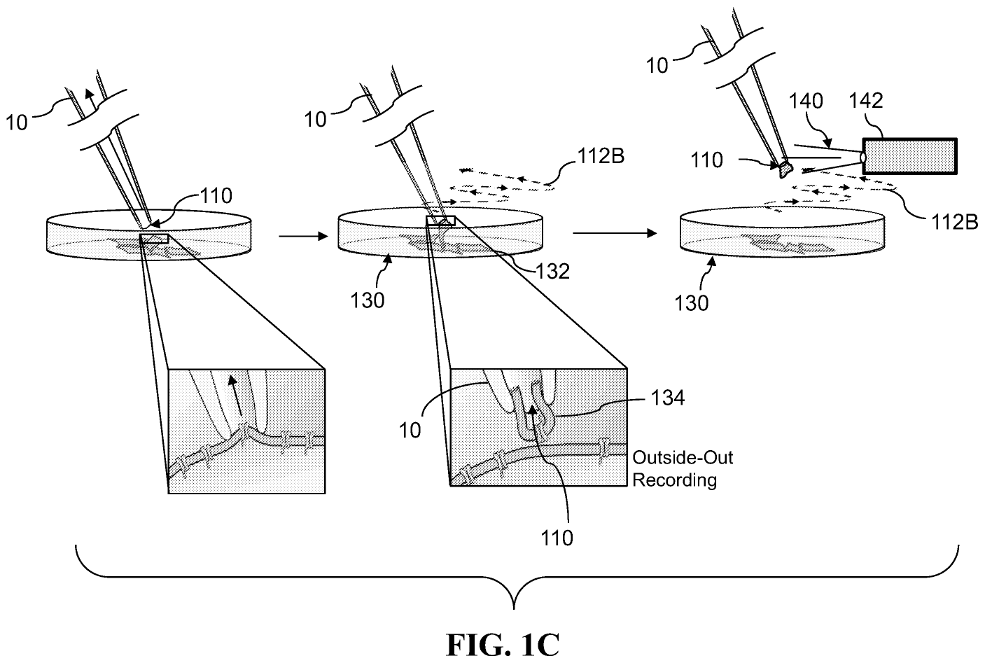

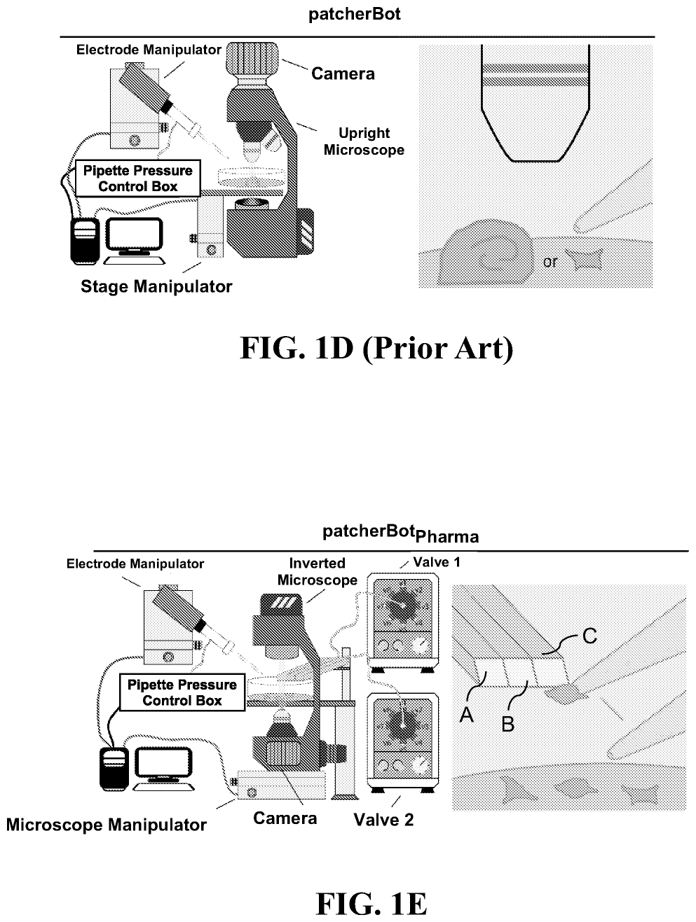

FIG. 1A is a schematic, pictorial illustration of a system 100 having a pipette tip 110 with an apertured surface at the distal end (shown more clearly in FIGS. 1B and 1C), and a controller 200 configured to isolate at least a portion of a biological sample target 132 from a sample of targets. The pipette tip 110 can be manipulable such that the pipette tip 110 is configured to move in the x-, y-, and/or z-plane, by a stage having a programmable linear motor 122, between a sample area 130 and an active agent exposure zone 140. The pipette tip 110 can be a hollow glass tube, such as a micropipette or a patch pipette that functions as an electrode. During a patch-clamp recording, the pipette tip 110 can be filled with an electrolyte solution and a recording electrode. With the pipette tip 110 movable within the sample area 130, the pipette tip 110 can be placed in contact with a biological sample target 132, such as a cell. A reference ground electrode (not shown) can be placed in the sample area 130 surrounding the biological sample, forming an electrical circuit between the recording electrode and the reference electrode with the biological sample target 132 in between. In examples described hereinbelow, the system 100 can be used for high-throughput drug screening, such as for performing patch-clamp recording of isolated cells, including cell-attached recording, whole-cell recording, inside-out recording, and/or outside-out recording methods. Alternatively, or in addition thereto, the system 100 may be used for measuring properties of cells that have been grown in a body and placed on a dish or for cells that have been grown in vitro, such as through microfluidics or other suitable methods, as well as for measuring properties of cells in vivo.

In some examples, the controller 200 can be configured to measure electrical properties of the biological sample target 132 at the pipette tip 110. In some instances, the electrical properties can be useful as a form of quality control measures of the system before, during, and after engaging a biological sample target. In particular, controller 200 can measure static electrical properties or a temporal series of electrical property measurements. Electrical properties can include one or more of a current, a capacitance, a resistivity, a conductivity, a temperature coefficient of resistance, a dielectric strength, thermoelectricity, or a combination thereof. Such electrical properties may be indicative of the characteristics of the sample target, such as the biological sample health, the quality of the high-resistance seal with the pipette tip, or the current flowing across a portion of the sample target, such as a cell membrane of a neuron.

In some examples, the electrical property measurement can be used to determine the quality or success of the isolated biological sample target 134 at the pipette-tip-to-cell interface. The electrical property measurements can be indicative of the high-resistance seal formed before, during, and after the pipette tip 110 engages with the isolated biological sample target 134. For instance, measuring the electrical resistance of the “patch” can provide feedback of the quality or success of such cell isolation to the system 100. In particular, when the pipette tip 110 engages a cell 132, the pipette tip 110 can perturb the cell 132 by a voltage to measure the difference in the current that flows through the cell 132 at the pipette-tip-to-cell interface. The difference in the voltage of the voltage pulse divided by the difference in the amount of current that flows through the cell and the pipette tip provides the resistance of the patch. If the resistance is in the Giga-ohm range, then it is deemed a high-resistance seal.

In some embodiments, the system 100 can achieve a whole-cell conformation such that patch-clamp recording of the biological sample target 132 at the pipette tip 110 includes the entire biological sample target 132, such as a cell. Alternatively, the system 100 may achieve a patch conformation such that patch-clamp recording of the biological sample target 132 at the pipette tip 110 includes only a portion of the biological sample target, such as a “patch” or a portion of the cell membrane of a cell. System 100 may be able to alternate between the different conformations depending on the experimental setup. In some examples, to achieve a whole-cell conformation, system 100 can apply a high negative pressure for short durations when the pipette tip is engaged with the biological sample target. The high negative pressure would pause or end when the electrical property measurement at the pipette tip indicates that a high-resistance seal has been created. Importantly, when the high negative pressure disrupts the membrane of a cell while the membrane is within the pipette tip, system 100 can maintain or moderately maintain the patch-clamp high-resistance seal. System 100 can receive feedback, for example, in the form of a capacitance measurement recorded at the pipette tip that may indicate the success of the gigaseal formation. A capacitance measurement that indicates formation of a high-resistance seal to a pre-determined value (>1 GQ) prior to initiating isolation of the cell may increase the quality and success of the process. In some examples, an increase in the capacitance of about 5 or more picofarad (pF) may indicate that a whole-cell conformation patch-clamp recording is achieved.

In addition, continued electrical property measurements can monitor for changes in the high-resistance seal such that the system 100 can be alerted if or when the electrical property measurement of the cell at the pipette tip falls below a predetermined cut-off level (<1000 MΩ) (e.g., about less than 900 MΩ, about less than 800 MΩ, about less than 700 MΩ, about less than 600 MΩ, about less than 500 MΩ, about less than 400 MΩ, about less than 300 MΩ, about less than 200 MΩ, about less than 100 MΩ, or any predetermined cut-off level in between, e.g., about less than 57 MΩ). As would be appreciated by those of skill in the art, the predetermined cut-off level may vary depending on various factors of the patch-clamp experiment. For instance, if a cell is adherent to a dish, the predetermined cut-off level may be set by system 100 as low as 50 MΩ, while the predetermined cut-off level of a lifted cell may be set by system 100 as low as 500 MΩ. Importantly, system 100 may be configured to adjust the predetermined cut-off level dependent on the cell type, cell environment, cell adhesion to such environment, patch-clamp recording style (e.g., whole-cell recording, cell-attached recording, inside-out recording, outside-out recording, and the like).

As shown in FIG. 1A, the pipette 10 can be attached at the proximal end to a pipette holder 12. The pipette tip 110 can be manipulable by a motorized stage 122 or from the pipette holder 12 by an internal mechanism within the pipette holder 12 such that the pipette tip 110 and the apertured surface can be moved about the sample area 130 and the active agent exposure zone 140, as well as to critical locations in contact with specified biological targets 132 within the sample area 130.

The active agent exposure zone 140 can include a fast perfusion system or delivery port 142 for distributing an active agent within the active agent exposure zone 140 without distributing or dispersing any active agent in or around the sample area 130. The delivery port 142 can include a number of solution reservoirs such as syringes or other solution containers with tubing to allow solution to flow from the respective reservoir to a manifold. Each reservoir can include a different active agent, or alternatively, the same active agent in varying concentrations between reservoirs. In addition, a solution in a first reservoir can be mixed with another solution in a second reservoir by connecting the tubes of each respective reservoir to a Y connector. Between the manifold and the reservoir, the fast perfusion system can include capillary glass barrels connected to the tubing and attached to a valve solution changer. Although the delivery port 142 is depicted with only a single flow in FIGS. 1B and 1C, the delivery port 142 can have a number of separate solutions flowing through barrels “A,” “B,” and “C,” as depicted in FIG. 1E. In addition, it is possible to have as many solutions as is experimentally necessary by using an appropriate manifold. For instance, for an experiment testing a dosing range of a mixture of three different active agents and a saline solution control, an appropriate manifold may include four barrels and the ability to mix different active agents to a desired concentration or dose using Y connectors. Alternatively, or in addition thereto, the various active agents may be premixed into the various solutions such that each premixed solution may be applied to the isolated biological sample target 134 as desired.

During a patch-clamp procedure, a user can insert the pipette 10 onto pipette holder 12 of the system 100 so that the pipette tip 110 at the distal tip of the pipette 10 approaches a biological sample target 132, such as a neuron or a cell. Upon the pipette tip 110, and specifically the apertured surface engaging with the cell, the system 100 can initiate a light aspiration through the pipette 10 by means of a pressure controller 160. To start performing patch clamping of the cell, the user can manipulate or the system 100 may automatically manipulate the vacuum pressure and/or pressure waveform pattern of the pressure controller 160 based on one or more input signals the electrode and the pipette tip 110 so that pipette tip 110 engages the cell.

The pressure controller 160 can be in communication with the pipette tip 110 for assisting in forming the high-resistance seal (>1 GQ), or “gigaseal” between the biological sample target 132 and the pipette tip 110. The pressure controller 160 can be configured to generate a negative (suction) pressure sufficient to cause the cell 132 to form the gigaseal for the various patch clamp variations ranging from inside-out, outside-out, cell-attached, or whole-cell, although in some examples, no negative suction pressure is required. The pressure controller 160 can be configured to create a suction pressure to assist in engaging a cell membrane during the patch-clamp procedure. In some examples, the pressure controller 160 can be a centrifugal pump, a rotary pump, a peristaltic pump, a roller pump, or other suitable form of pump known in the art. The pressure controller 160 can be capable of producing pressures from −1000 to +1000 mbar (e.g., from about −1000 mbar to about −900 mbar, from about −900 mbar to about −800 mbar, from about −800 mbar to about −700 mbar, from about −700 mbar to about −600 mbar, from about −600 mbar to about −500 mbar, and from about −500 mbar to about −400 mbar, from about −400 mbar to about −300 mbar, from about −300 mbar to about −200 mbar, from about −200 mbar to about −100 mbar, and from about −100 mbar to about 0 mbar, from about 0 mbar to about +100 mbar, from about +100 mbar to about +200 mbar, from about +200 mbar to about +300 mbar, from about +300 mbar to about +400 mbar, from about +400 mbar to about +500 mbar, and from about +500 mbar to about +600 mbar, from about +600 mbar to about +700 mbar, from about +700 mbar to about +800 mbar, from about +800 mbar to about +900 mbar, and from about +900 mbar to about +1000 mbar, and any pressure in between, e.g., from about −528 mbar to about +133 mbar). In addition, pressure can be applied in discrete positive pressure states with a high positive pressure ranging from about +1000 mbar to about +2500 mbar (and any pressure in between) and a discrete low positive pressure ranging from about +50 mbar to about +900 mbar (and any pressure in between). Similarly, the pressure can be applied in discrete negative pressure states with high negative pressure ranging from about −500 mbar to about −1000 mbar (and any pressure in between) and a discrete low negative pressure ranging from about −250 mbar to about 0 mbar (and any pressure in between).

In the configuration shown in FIG. 1A, the controller 200 is connected, by one or more cables 201 to the system 100. Controller 200 includes a processor 46 that, in conjunction with a tracking module 220 and a manipulator assembly 120, can determine location coordinates of the pipette 10 and/or the pipette tip 110. Controller 200 can additionally include an isolation module 230, a tracking module 220, an electrophysiology module 250, and optionally a cleaning module. As used herein, the described modules are a collection of software and/or hardware.

In the tracking module 220, the controller can position the pipette tip 110, by the motorized stage 122, in close proximity to a biological sample target 132 of interest. Location coordinates of the pipette tip 110 can be determined based on one or more high sensitivity optical cameras or microscopes (not depicted in FIG. 1A). The selection of biological sample targets 132 can be achieved through the use of the optical camera or microscope. Alternatively, or in addition thereto, the location coordinates of the pipette tip 110 can also be determined based on an electromagnetic position sensor output signal provided from the distal portion of the pipette 10 when in the presence of a generated magnetic field.

In the isolation module 230, the controller 200, in conjunction with the manipulator assembly 120, motorized stage 122, and/or pressure controller 160, can form a high-resistance seal, or gigaseal with at least a portion of the biological sample target 132. After tracking the biological sample target 132 of interest, the pipette tip 110 in suitable position can form a gigaseal with such target 132. Next, the isolation module 230 can either lift a biological sample target 132 or pull at least a portion of the biological sample target 132, such as a cell, in a whole-cell conformation as shown in FIG. 1B, or an outside-out patch conformation as provided for illustrative purposes in FIG. 1C. In the isolation module 230, the controller can be configured to move the biological sample target 132 (either whole cell, or a patch of such cell) with at least one of a pre-determined pattern 112, a pre-determined speed or rate of motion, and/or a pre-determined pressure such that the gigaseal remains intact between the pipette tip 110 and the now isolated biological sample target 134. In addition, or alternatively thereto, the system 100 can receive feedback from the pipette tip based on a change in the high-resistance seal between the pipette tip 110 and the isolated biological sample target 134. In particular, a change in the high-resistance seal can be indicative of a potential loss of the high-resistance seal between the pipette tip 110 and the isolated biological sample target 134. System 100 can receive such feedback and modify at least one of the pre-determined pattern of motion 112, the pre-determined speed, and/or the pre-determined pressure to prevent the loss of the high-resistance seal between the pipette tip 110 and the isolated biological sample target 134.

As depicted in FIG. 1B, for whole-cell conformation, the pre-determined pattern 112 can include a spiral motion 112A in combination with a suction on the pipette tip 110 such that the isolation module path includes spiraled discrete segments to routinely lift the biological sample target 132 with an increased success of maintained gigaseal between the pipette tip 110 and the target 132. As shown in FIG. 1C, for outside-out patch conformation, the pre-determined pattern 112 can include an arc motion 112B in combination with little to no suction on the pipette tip 110 such that the isolation module path includes arced discrete segments to lift a patch of the biological sample target 132 routinely and selectively with an increased success of maintained gigaseal between the pipette tip 110 and the target 132.

The pre-determined pattern can include one or more of a spiral pattern, a helical pattern, a wave pattern, a square pattern, a triangle pattern, a sawtooth pattern, a reciprocating pattern, an oscillating pattern, a rotary pattern, a linear pattern, a translatory pattern, a rectilinear pattern, a curvilinear pattern, a parabolic motion, a circulatory pattern, a vibratory pattern, a periodic pattern, and combinations thereof. For instance, in the z-plane, the controller 200 A spiral pattern is a motion in which the object moves from an initial position moves along a curved path that increases in diameter from the initial position and the object moves along the z-plane. A helical pattern is a motion in which the object moves from an initial position along a curved path that remains constant as the object moves along the z-plane. A wave pattern can include a motion in which the object moves along a curved path for only a portion of the curve.

The pre-determined pattern can also be 4-dimensional in that time can be adjusted throughout the translatory motion. For instance, quick motions may lead to a change in the high-resistance seal indicative of a disruption of the seal between the pipette tip and the cell.

Translatory motion is the motion in which the object moves in the same distance at the same time. Rectilinear and Curvilinear motions are the subcategories of translational motion. Rectilinear motion, also known as linear motion, is said to be present if the object moves along a straight line. Curvilinear motion occurs when the object moves along a curved path. Circular motion or circulatory motion is the movement of objects along the circular path, which means along the path whose every boundary point is equidistant from a fixed point, i.e., center. Rotatory motion is said to exist if the object rotates or spins about its axis. In oscillatory motion, an object tends to repeat certain movements in the same pattern again and again, until it is acted upon by some external force. Some objects display a rapid back and forth motion about a fixed point when their equilibrium condition is disrupted. This type of motion is known as vibratory motion. Periodic motion is motion that will be repeated after a certain period of time. Reciprocating motion and vibratory motion are very closely related to each other, but there is a small exception that reciprocating motion necessarily involves up-and-down or back-and-forth motion.

Referring back to FIG. 1A, the processor 46 of the controller 200 can also include a testing module 240, that, in conjunction with the manipulator assembly 120, motorized stage 122, pressure controller 160, and/or active agent perfusion assembly 150, the controller 200 can translate the isolated biological sample target 134 from the sample area 130 to the active agent exposure zone 140.

In the electrophysiology module 250, the processor 46 of the controller 200 can be configured with the manipulator assembly 120, motorized stage 122, pressure controller 160, and/or active agent perfusion assembly 150 to expose (via delivery port 142) at least a portion of the isolated biological sample target 134 sealed to the pipette tip 110 with one or more active agents in the active agent exposure zone 140. In particular, when the isolated biological sample target 134 is within the active agent exposure zone 140, in the electrophysiology module 250, the controller can be initiated to study ionic currents in the isolated biological sample target 134 before, during, and/or after exposure to one or more active agents.

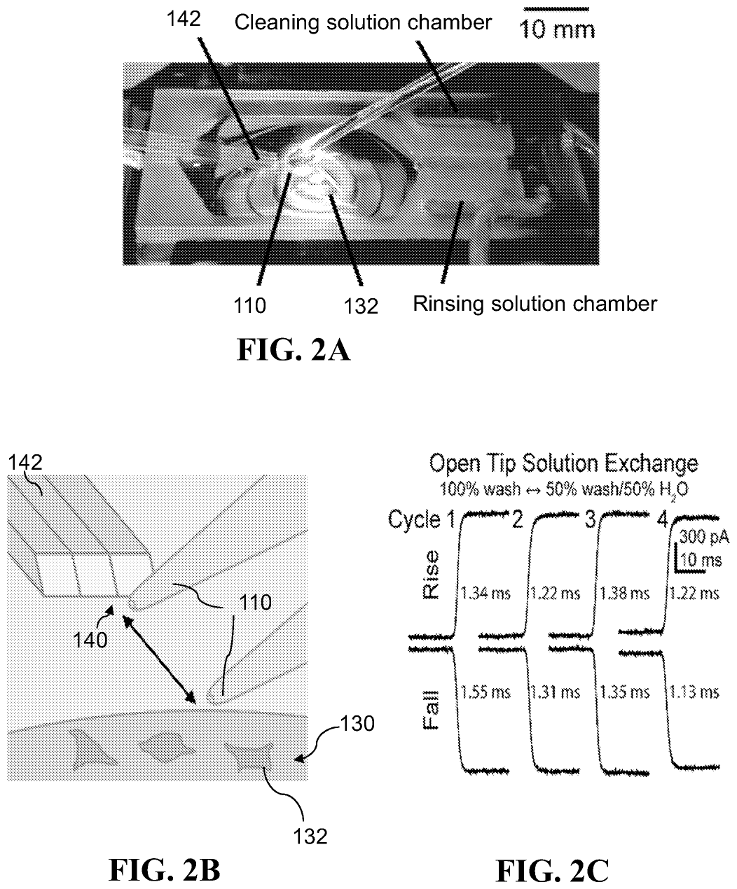

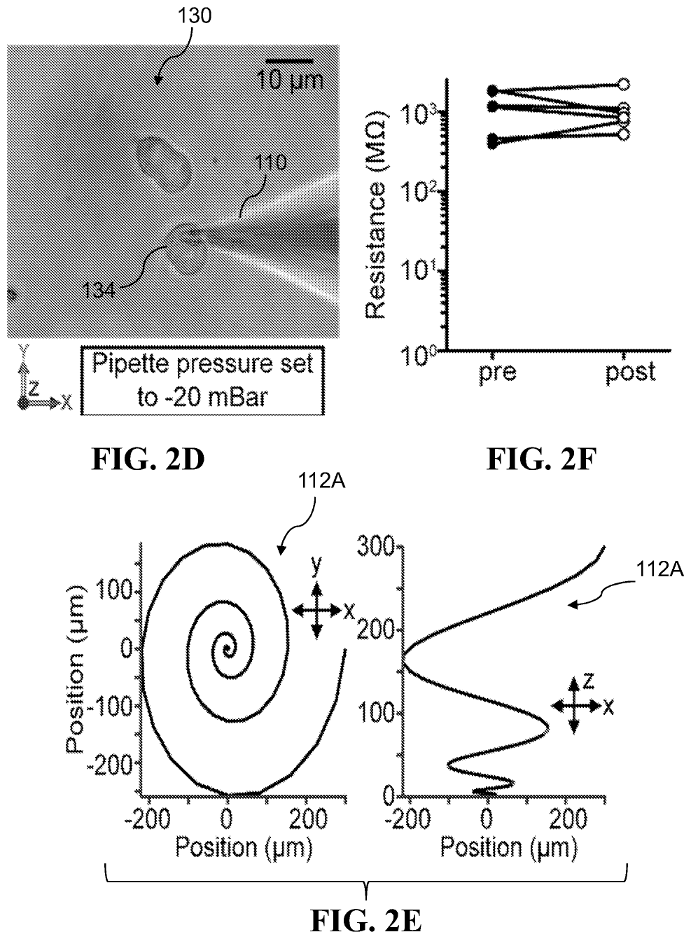

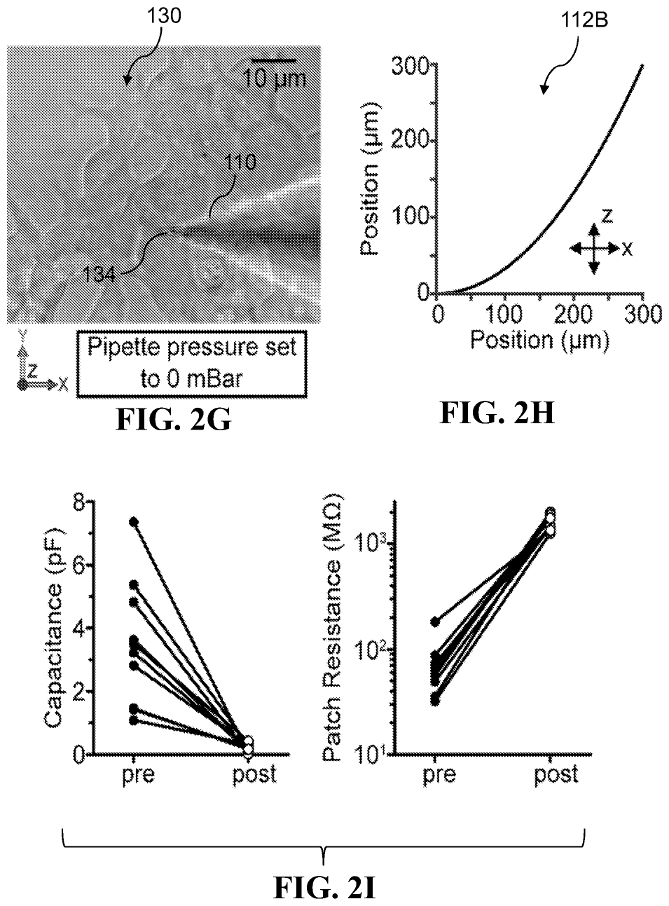

Although not depicted in FIG. 1A, the processor 46 of the controller 200 can optionally include a cleaning module. The cleaning module can include a cleaning solution chamber containing a cleaning solution and a rinse solution chamber containing rinse solution are situated near the sample area 130 (as depicted in FIG. 2A). Pressure is applied via the pressure controller 160 to the pipette tip 110 through the pipette 10. The three-axis motorized stage 122 can move the pipette 10 and the pipette tip 110 between the cleaning solution chamber, the rinse solution chamber, the sample area 130, and the active agent exposure zone 140.

Alternatively, or in addition thereto, the controller 200 while in at least one of the isolation module 210, tracking module 220, or electrophysiology module 250 can be configured to adjust the speed, pattern, pressure, waveform pattern, or other attributes of the motorized stage 122 and pressure controller 160 based on feedback received from pipette tip regarding the electrical property measurements of the isolated biological sample target 134.

Processor 46 may include real-time noise reduction circuitry 50 typically configured as a field programmable gate array (FPGA), followed by an analog-to-digital (A/D) signal conversion integrated circuit 52. The processor 46 can be programmed to perform one or more algorithms and uses circuitry 50 and circuit 52 as well as features of modules to enable the user to perform the patch-clamp procedure.

The controller 200 also includes an input/output (I/O) communications interface 54 that enables the controller 200 to transfer signals from, and/or transfer signals to the pipette tip 110. In an example implementation, the I/O communication interface 54 may provide functions for rendering video, graphics, images, text, other information, or any combination thereof on a display interface 64. In one example, the communication interface 54 may include a serial port, a parallel port, a general-purpose input and output (GPIO) port, a game port, a universal serial bus (USB), a micro-USB port, a high-definition multimedia (HDMI) port, a video port, an audio port, a Bluetooth port, a near-field communication (NFC) port, another like communication interface, or any combination thereof. In one example, a display interface 64 may be operatively coupled to a local display, such as a touch-screen display associated with a mobile device. In another example, the display interface 64 may be configured to provide video, graphics, images, text, other information, or any combination thereof for an external/remote display that is not necessarily connected to the mobile computing device. In one example, a desktop monitor may be utilized for mirroring or extending graphical information that may be presented on a mobile device. In another example, the display interface 64 may wirelessly communicate, for example, via the network connection interface such as a Wi-Fi transceiver to the external/remote display.

In some instances, as described above, the pressure controller 160 may be controlled by the controller 200 via the I/O communications interface 54. The controller 200 may be operated by the user to adjust the speed, pressure, waveform pattern, or other attributes of the pressure controller 160 during the patch-clamp procedure. Digital TTL signals from pressure controller 160 can switch the individual valves of pressure controller 160 to change which pressure is applied to the pipette tip 110 during use. The controller 200 may receive input from the user to control the pressure controller 160 via the input devices 68 or on a separate aspiration pump input via dials, touch screens, buttons, or levers for adjusting pump speed or another pump variable. In some examples, the controller 200, manipulator assembly 110, active agent perfusion assembly 150, and pressure controller 160 may be integrated as a single device.

During the patch-clamp procedure, processor 46 can present the procedure data 62 to the user on a display interface 64, and store data representing the procedure data 62 in a memory 66. Memory 66 may include any suitable volatile and/or non-volatile memory, such as random-access memory or a hard disk drive.

In some examples, the user can manipulate the procedure data 62 using one or more input devices 68. In alternative examples, display interface 64 may include a touchscreen that can be configured to accept inputs from the user in addition to presenting any procedure data 62. It is understood that a user interface can be accessed on various devices (e.g., laptop computer, tablet, etc.) and be embodied in various forms. As desired, implementations of the disclosed technology may include a computing device with more or less of the components illustrated in FIG. 1A. It will be understood that the architecture of the controller 200 is provided for example purposes only and does not limit the scope of the various implementations of the present disclosed systems, methods, and computer-readable mediums.

FIGS. 1D and 1E provide a comparison of patcherBot vs patcherBot_{Pharma}. In particular, FIG. 1D is a schematic of a prior implementation of the presently disclosed technology. In the prior implementation, the system is assembled from an upright microscope, high sensitivity camera, custom pressure control box, quasi-4 axis electrode manipulator, and a motorized stage. In the present implementation illustrated in FIG. 1E, an example system is assembled from an inverted microscope, high sensitivity camera, custom pressure control box, quasi-4 axis electrode manipulator, a motorized microscope manipulator, two solution valves, and a solution exchange manifold.

FIG. 2A is an image of an example system 100, showing the pipette tip 110 at the sample area 130, with active agent perfusion assembly 150. FIG. 2B is a schematic, pictorial illustration of the distance the pipette tip 110 may translate a biological sample target 132 between the sample area 130 and the active agent exposure zone 140 during use. Repeatability of the physical manipulations require for fast-solution exchange electrophysiological experiments. FIG. 2C is a current recording versus time testing the ability of a pipette tip to return to a critical location. Open-tip solution exchange times, using piezo-electric translator, across many repeated experimental cycles (cell locations, solution manifold interface, cleaning/wash bath).

Turning now to FIGS. 2D through 2E for a whole-cell patch-clamp configuration, FIG. 2D is an image of the pipette tip 110 in position to isolate a biological sample target (a cell) 132. In particular, the image depicts an isolated cell 134 in the whole-cell conformation before lifting (isolated cell may be more reliably lifted than those with cellular processes to adjacent cells). FIG. 2F provides a resistance plot showing a high-resistance seal is robustly maintained during the lifting process. FIG. 2E provides an example spiral motion path that can be used to lift an isolated cell 134 from the sample area 130. During the lifting process, the resistance can be monitors to ensure the high-resistance seal is robustly maintained during the lifting process.

FIGS. 2G through 2I, for a patch pulling procedure, FIG. 2G provides an image of a cell in the whole-cell conformation before pulling a patch, such as for an outside-out or an inside-out recording. FIG. 2H provides an example arc path (100 discrete segments) that may be employed to pull outside-out patches. FIG. 2I provides resulting capacitance and resistance plots of the example arc path, showing successful high-resistance and low-capacitance outside-out patches. It is anticipated that the low resistance prior to pulling the outside-out patches is due to electrical connections due to gap-junctions between multiple cultured cells in physical contact with one another.

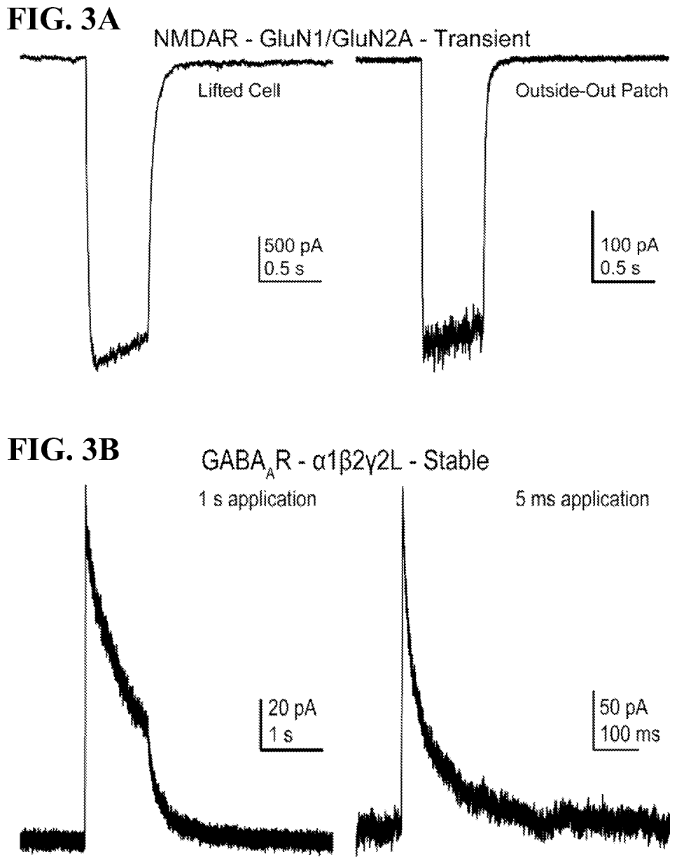

FIGS. 3A and 3B provide exemplary fast-solution exchange electrophysiological experimental results using the system 100. In particular, FIG. 3A shows N-methyl-D-aspartate receptor (NMDAR) responses from transiently transfected HEK cells stimulated by 100 μM glutamate and 30 μM glycine. Recordings are from a lifted whole cell (left) and an outside-out patch using a 4 MΩ electrode (right), at −60 mV in 0 mM Mg²⁺. FIG. 3B shows γ-aminobutyric acid type A receptor (GABA_{A}R) responses from stably transfected HEK cells (α1β2γ2 L) stimulated by 1 mM GABA. Recordings are from a lifted whole cell (left, 1 s application) and an outside-out patch (right, 5 ms application).

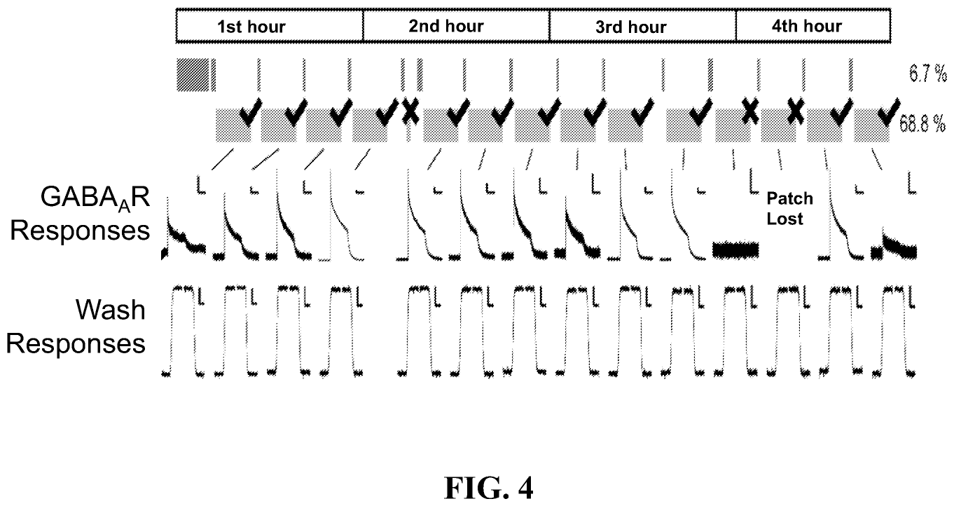

FIG. 4 provides a representative experimental timeline of the system 100 operation with recording results and plots of capacitance and resistance for each electrophysiological experiment conducted during the timeline. Representative experimental timeline of patcherBot_{Pharma} operation. At the top of FIG. 4, a timeline illustrates the experimental progress. The time periods of operator interaction with the patcherBot_{Pharma} (approximately 6.7% of the total recording duration) and recording duration are shown as gray boxes throughout the timeline. The check marks throughout the timeline indicate a successful electrophysiological experiment recording outcome while the cross marks indicate a failed or unrecordable outcome. Below the timeline, FIG. 4 shows plots of GABA_{A}R responses (1 mM GABA, 1 s application) from all successful outside-out patches pulled. Scale bars indicate 20 pA and 0.5 s. Below the plots of GABA_{A}R responses, FIG. 4 provides post-experiment open-tip position validation utilizing a 50% H₂O/50% wash solution. Scale bars indicate 200 pA and 20 ms. The average (±SD [Range]) 20-80 rise and fall times for piezoelectric jumps were 3.06±0.78 [1.30 4.11] and 3.56±0.32 [2.27 6.55].

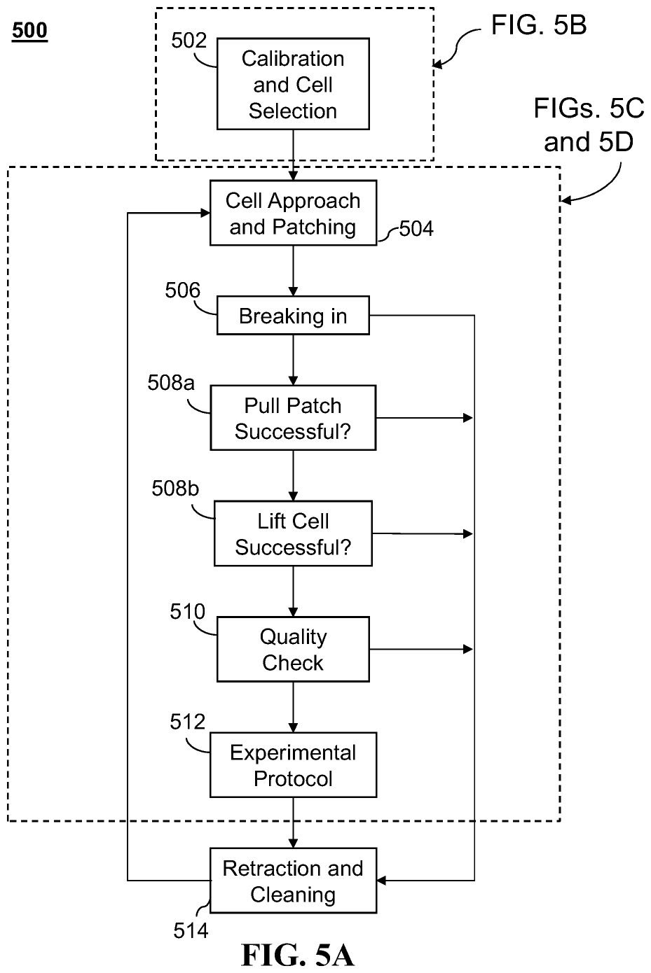

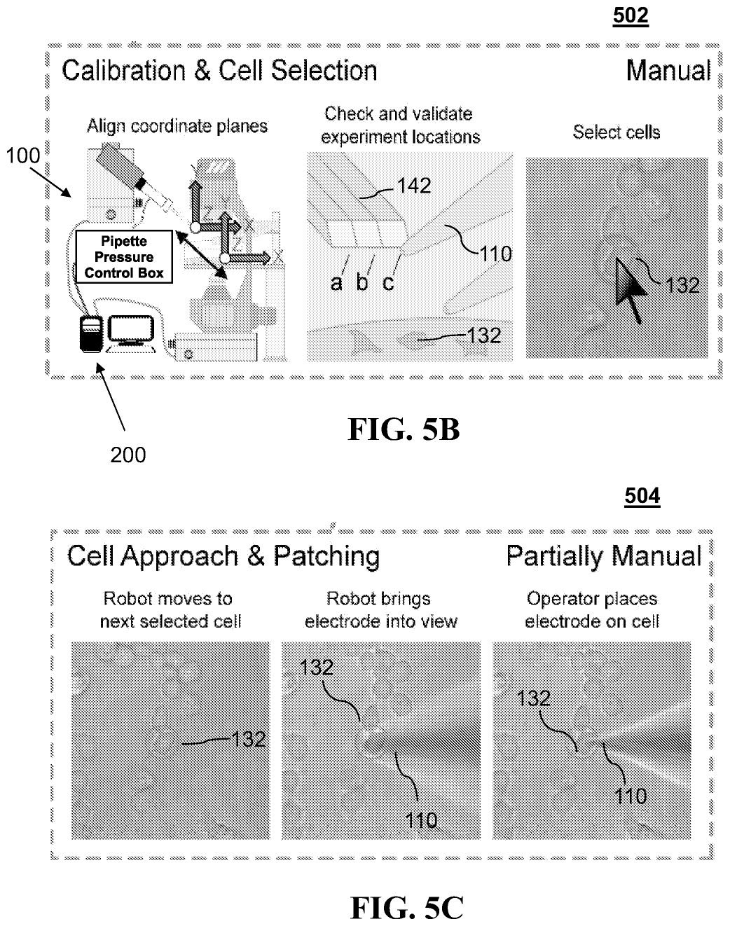

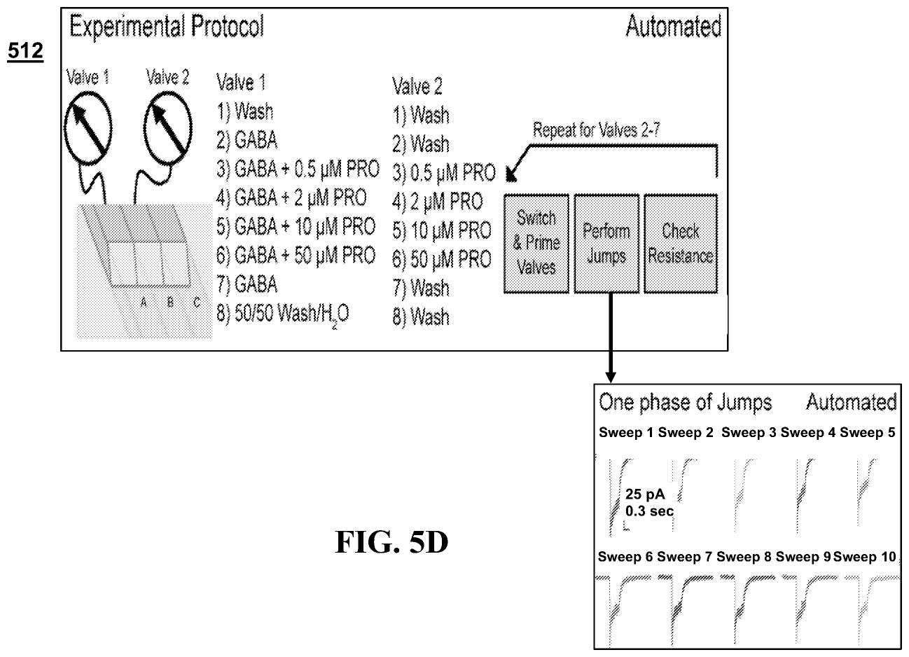

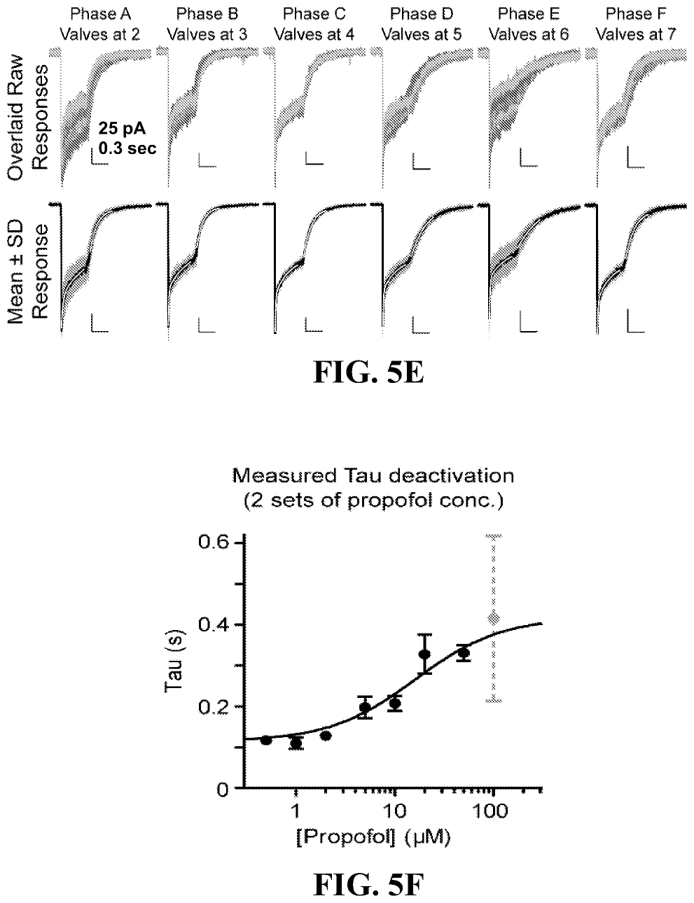

FIGS. 5A through 5D provides various steps system 500 can perform to achieve a time-constant concentration response of deactivation of a cell receptor exposed to a drug. In an exemplary case study, GABA_{A}R propofol deactivation time-constant concentration response was studied to establish that the system 500, the patcherBot_{Pharma}, has the capability to collect pharmacological data at an accelerated rate. FIGS. 5A through 5D the individual steps in a flowchart illustrating the patcherBot_{Pharma} operation, timing, and success rate. After the one-time calibration and cell selection step 502, system 500 loops through and serially records from the selected cells in steps 504 through 514. Quality control measures are in place at steps 506, 508a, 508b, and 510 to terminate the current experiment and continue to the next iteration, as shown in FIG. 5A. System 500 can perform calibration and cell selection 502 manually such that the user selects one or more cells within a biological sample. In some instances, calibration and cell selection 502 can be performed automatically. For instance, calibration can be pre-programed and stored in memory 66 of controller 200. Alternatively, or in addition thereto, calibration can be automatically sensed by a combination of the manipulator assembly 120 and automated control of a microscope. Cell approaching can be achieved in approximately 5 minutes or less (e.g., approximately 4 minutes or less, approximately 3.5 minutes of less, approximately 3 minutes or less, approximately 2.5 minutes of less, approximately 2 minutes or less, approximately 1.5 minutes of less, approximately 1 minute or less, approximately 45 seconds or less, approximately 30 seconds or less, approximately 15 seconds or less, or any time range in between, e.g., approximately 3.28 minutes or less).

A more detailed depiction of the calibration and cell selection step 502 is shown in FIG. 5B. The calibration and cell selection step can include alignment of the coordinate systems of the electrode and microscope, ensuring the saved locations of the solution manifold are correct, and then selecting a set of cells for recording. In some instances, between 7-12 cells can be selected for recording, however more cells can be selected for larger experiment protocols using system 500. For instance, more than 7 cells can be selected to be serially recording (e.g., 8 or more cells, 10 or more cells, 15 or more cells, 20 or more cells, 50 or more cells, 100 or more cells, or 500 or more cells).

After cell selection and calibration, the system 500 can automatically approach the cell at step 504 and initiate forming a high-resistance seal or gigaseal. The cell approach and patching step 504 can be the beginning of each loop (coinciding with an auditory signal, so the operator need not always be present). In some instances, step 504 starts when the system 500 translates the stage to the next cell selected, then the electrode is brought to a position just above (approximately 100 μm) the cell. In some instances, the system 500 can include a robot that can automatically move the pipette 10 and pipette tip 110 to the selected cell, as shown more clearly in FIG. 5C. The user may also provide manual input into the motion of the pipette 10 and pipette tip 110, such as by lowering the electrode to the optimal position on the cell. In addition, the user may have the option of manually seal and break in, or can elect to have the system 500 conduct the process of sealing and breaking in.

Once the pipette tip 110 is placed on the selected cell, the system 500 can initiate the patching step and breaking into the cell target at step 506. As described supra, quality control measures are in place at steps 506, 508a, 508b, and 510 to terminate the current experiment and continue to the next iteration. If any quality measures indicate that a cell target is no longer adequately sealed with the pipette tip 110 (i.e., the high-resistance seal measurement falls below the predetermined cut-off level as described supra), the system 500 will move to retraction and cleaning in step 514 in order to reinitiate the loop and approach the next cell (step 504) from the cell selection (step 502).

After quality control measures verify an appropriate seal between the pipette tip 110 and the cell target, system 500 can initiate the experimental protocol at step 512. A more detailed look at the experimental protocol step of the system 500 is shown in FIG. 5D. In some examples, a set of 6 solutions were used during each experiment (2 control and 4 propofol solution sets). Each phase of each experiment would start with the valves changing to the next set to be tested, with a wait step to allow for the solutions to be primed, followed by the collection of 10 replicates of the intended jump protocol.

FIG. 5E provides the results from one experiment (all phases), showing all replicates (top) and the average (±SD, shown by shaded grey area) response. The desensitization and deactivation of all recordings were fitted simultaneously and are depicted on the averaged responses (white line). FIG. 5F shows the relationship between the average (±SEM) deactivation tau and propofol concentration is shown and fitted with the Hill equation. The 100 μM propofol response was omitted from fitting due to the reduced response amplitude as a result of the enhanced desensitized state in the presence of such a high concentration of propofol.

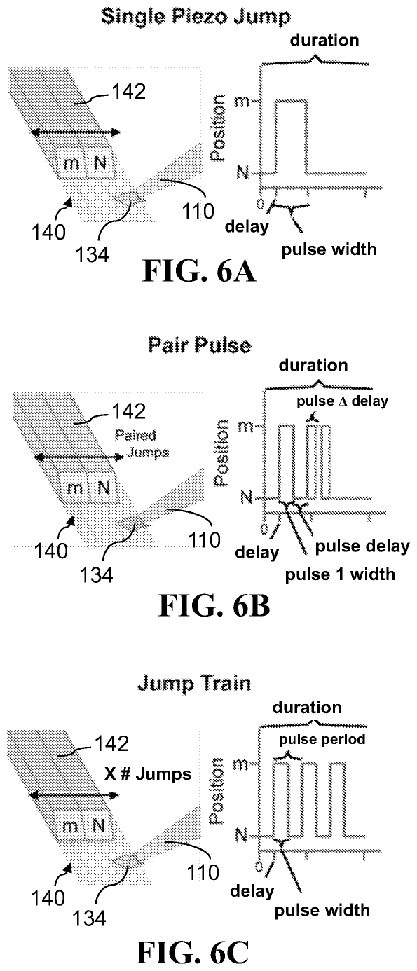

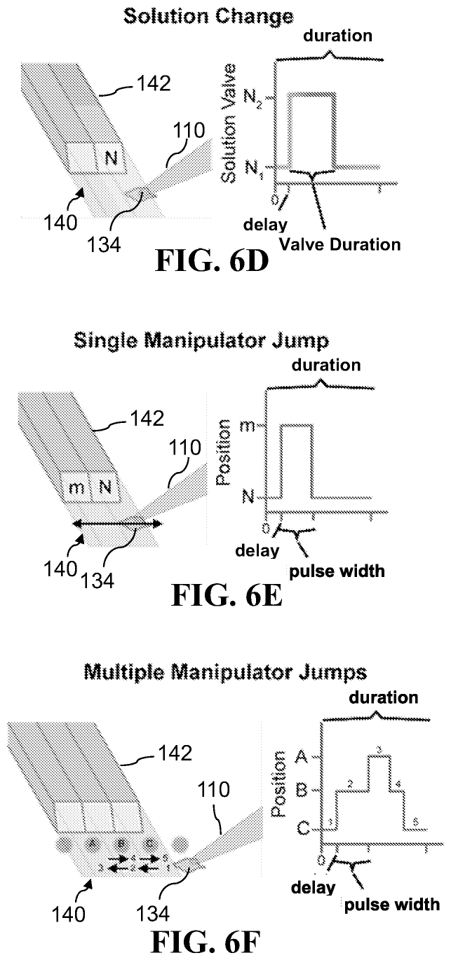

FIGS. 6A through 6F are schematic, pictorial illustrations of example solution exchange protocols programmed into the system 100. Solution exchange protocols programmed into the system 100 can utilize either movement of the piezo-mounted manifold 134, or movement of the manipulator holding the pipette 110. As shown in FIGS. 6A through 6C, a piezoelectric translator can be controlled by a filtered voltage pulse signal, in either individual pulses (FIG. 6A), paired pulses with an optional increasing delay (FIG. 6B), or a train of applications with variable pulse width and period (FIG. 6C). In FIG. 6D, a slow solution exchange experiment can be performed where the solution valve is switched at a specific time during the experiment to record the slow transition of the application of a second solution. In FIG. 6E, a smooth motion from the electrode manipulator can be used to perform a similar solution application to FIG. 6A, but with a broader range of motion allowing multiple barrels to be used. FIG. 6F illustrates that any number of manipulator transitions can be combined to produce complex solution applications.

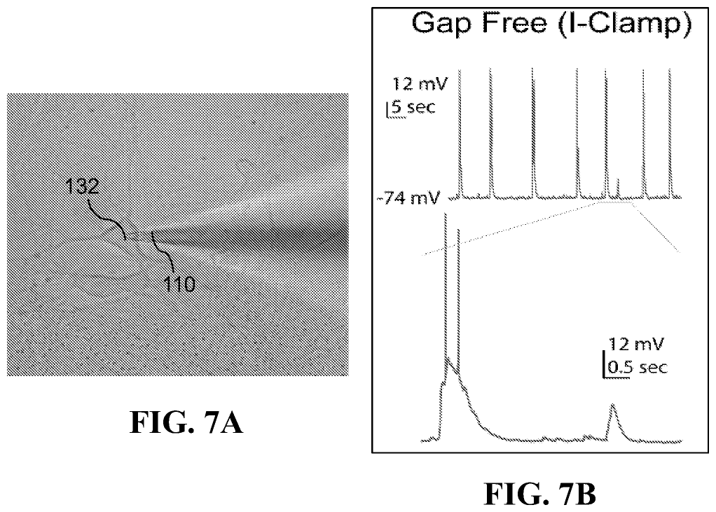

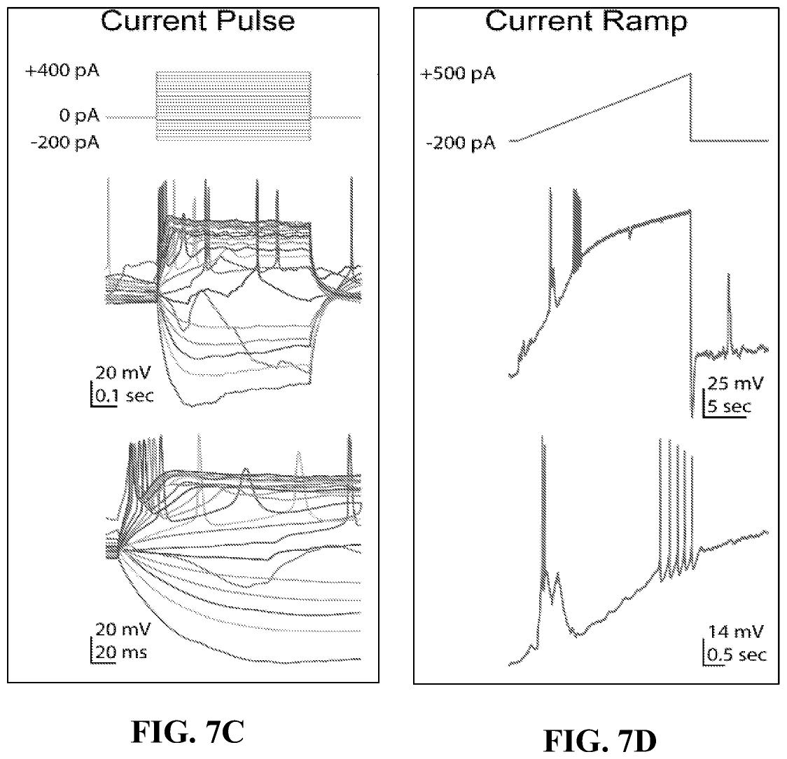

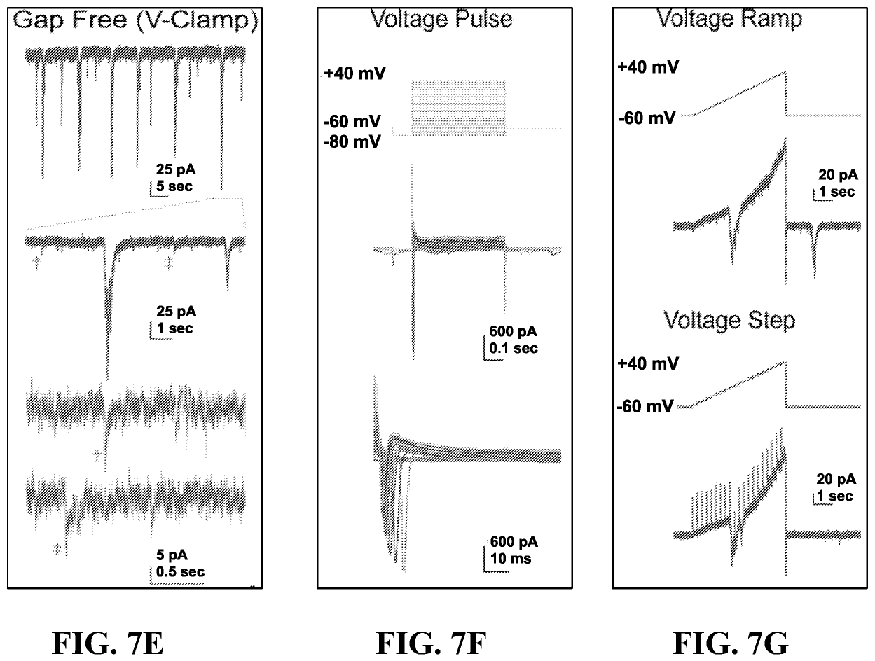

FIGS. 7A through 7G provide an image of a pipette tip 110 contacting a cell 132 and example resulting voltage-clamp protocol and current-clamp protocol examples that can be accomplished by and/or programmed into the system 100. FIG. 7A is a bright-phase image of DIV 14 cortical neuron during a recording and FIG. 7B is an exemplary current-clamp gap-free recording (I_{injection}=0 pA), showing spontaneous network activity, bursting, and the resting membrane potential (−74 mV). FIGS. 7C and 7D are current-pulse protocol and a current-ramp protocol, illustrating the amount of injected current is required to induce an action potential. FIG. 7E is an exemplary voltage-clamp gap-free recording (V_{holding}=−60 mV), showing spontaneous network activity and even putative spontaneous events (pink staffs). FIG. 7F illustrates that a two-part voltage-pulse protocols can elucidate voltage-gated channel responses. As shown in FIG. 7G, voltage-ramp and step protocols can also be applied to measure membrane properties.

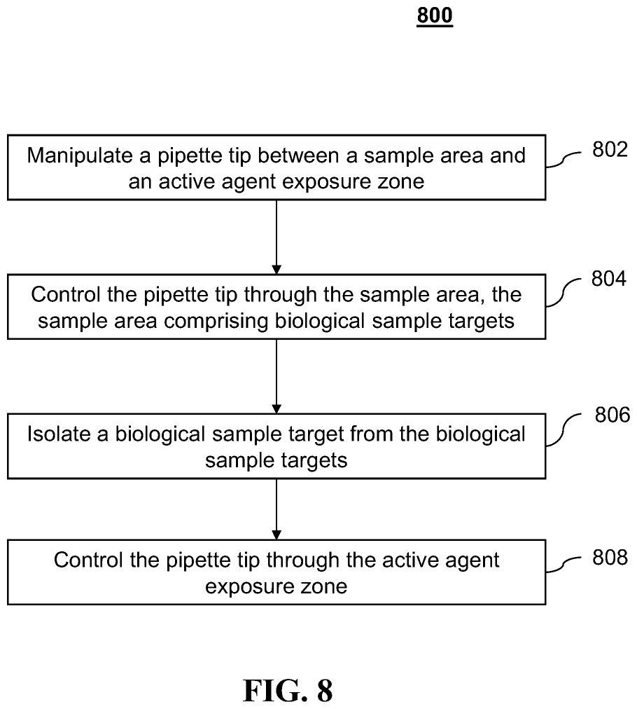

FIG. 8 is a flowchart illustrating method 800 for detecting an effect of an active agent on a cell using the system 100. Method 800 can include manipulating 802 a pipette tip between a sample area and an active agent exposure zone, using the controller and systems described supra. In addition, method 800 can further include controlling 804 the pipette tip through the sample area. The sample area can contain one or more biological sample targets, such as cells or subjects for in vivo patch-clamp recording. In addition, method 800 can include isolating 806 a biological sample target from the biological sample targets. After isolating a biological sample target, method 800 can include controlling 808 the pipette tip through the active agent exposure zone. Method 800 can end after step 808 or can alternatively add another step of cleaning the pipette tip and beginning again at step 802 with a new biological sample target.

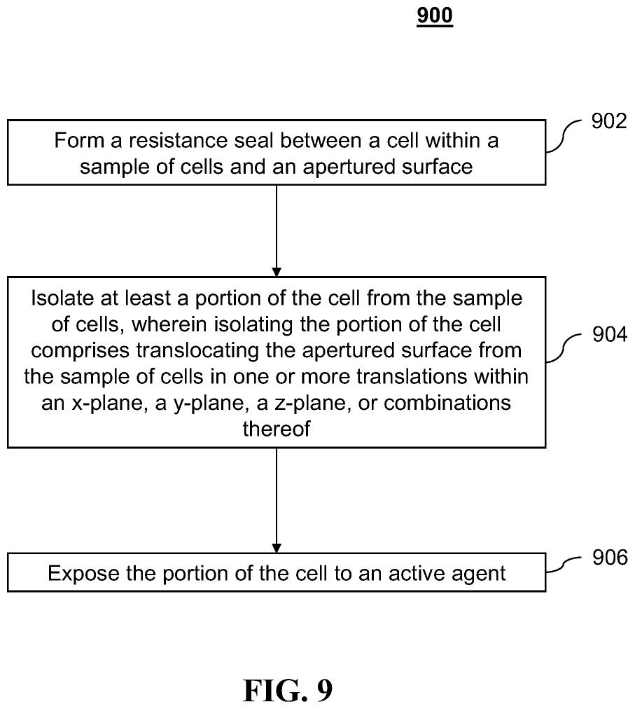

FIG. 9 is a flowchart illustrating method 900 for detecting an effect of an active agent on a cell using the system 100. Method 900 can include forming 902 a high-resistance seal between a cell within a sample of cells and an apertured surface. The high-resistance seal can be formed with additional steps such as applying a light suction through the apertured surface as described supra or may form without any additional steps. Method 900 can also include isolating 902 at least a portion of a cell from the sample of cells. Isolating 902 the cell can include translocating the apertured surface from the sample of cells in one or more translations within an x-plane, a y-plane, a z-plane, or combinations thereof. As described supra, isolating 902 may also include translocating the apertured surface as a factor of speed or rate of translocating in addition to a pressure applied to the portion of the cell through the apertured surface. In addition, method 900 may include exposing 906 the cell to an active agent. Method 900 may end after step 906 or may alternatively add additional steps such as, for example, recording of electrical properties of the portion of the cell sealed to the apertured surface or cleaning the apertured surface and beginning again at step 902 with a new cell.

The following examples further illustrate aspects of the present disclosure. However, they are in no way a limitation of the teachings or disclosure of the present disclosure as set forth herein.

### EXAMPLES

The invention presented herein can be an automated intracellular pharmacological electrophysiology robot, patcherBot_{Pharma}, that substantially improves throughput and reduces human time requirements in pharmacological patch-clamp experiments. The robotic system includes millisecond fluid exchange handling and can perform highly efficient ligand-gated ionotropic receptor experiments. The patcherBot_{Pharma} is built using a conventional patch-clamp rig, and the technical advances shown in this work greatly accelerate the ability to conduct high-fidelity pharmacological electrophysiology.

Here, a prior implementation of the disclosed technology is shown in FIG. 1D. The new implementation shown in FIG. 1E enables automated intracellular pharmacological electrophysiology of an isolated cell or patch, such that the rest of the sample tissue is not exposed to pharmacological active agents that would impair the electrophysiological recording of cells not being recorded. The “patcherBot_{Pharma}” can perform pharmacological concentration-response experiments and can record ligand-gated ionotropic receptor responses to fast agonist exposure (ms exchange time) with automated control of the microscope, bath solution, a solution manifold, and a piezoelectric translator. A high-throughput rate of the patcherBot_{Pharma} unattended is observed, with further improvements using minimal operator assistance. The capabilities of the patcherBot_{Pharma} are demonstrated by replicating a conventional dataset substantially faster—with considerably less human effort than demonstrated previously. The increased efficiency enabled by the patch-clamp electrophysiology system disclosed herein creates the potential to address scientific questions that were previously considered impractical because of large, time-consuming requirements needed to complete data acquisition using conventional approaches.

### Example 1: PatcherBot_{Pharma} Hardware and Software

The patcherBot_{Pharma} is built on a standard inverted microscope (Axiovert 200, Ziess) to allow for clearance of the recording electrode and solution handling manifolds. Standard, three-axis micromanipulators were used to translate the recording electrode (PatchStar, Scientifica) and the microscope (Motorized XY Stage [UMS] with Z-focus module, Scientifica). A high sensitivity camera (Retiga Electro, QImaging) is used for computer vision. Electrode pressure was controlled using a custom control box that regulates house-air line to deliver −700 to +1000 mbar using an inline venturi tube (SMC), solenoid valve (Parker Hannifin), and a digital air regulator (ProportionAir) controlled by an Arduino Uno for rapid pressure switching. A three-barreled, square cross-section solution manifold (3SG700-5, Warner Instruments) attached to a piezoelectric translator (Burleigh Instruments) was used for cell perfusion. Barrels of the solution manifold were connected to 8-valve solution changers (Hamilton Modular Valve Positioner). Custom LabVIEW code (National Instruments) integrating manipulators (electrode and microscope), camera view of the microscope stage, pressure control box, piezoelectric translator, and solution valves was implemented to control the rig and enable automated experimentation. Communication between the computer and the amplifier, piezoelectric translator and solution changers was achieved using a DAQ (BNC-2110, National Instruments) with several analog and digital interfaces.

### Example 2: Transiently Expressing HEK Cells

HEK-293 cells (CRL 1573, ATCC; hereafter HEK cells) and a stable GABA_{A}R-expressing cell line were cultured in DMEM (Cat #10566016, ThermoFisher Scientific) supplemented with 10% fetal bovine serum (FBS), 10 U/ml penicillin, and 10 lag/ml streptomycin and maintained at 5% CO₂ in a 37° C. incubator. For use on the electrophysiology rig, heterologous cells were plated on poly-D-Lysine (PDL) coated glass coverslips (0.1-0.5 mg/mL, Warner Instruments). Transiently transfected recombinant receptors were expressed from complementary DNA (cDNA) encoding rat GluN1-1a (hereafter GluN1, U08261) and GluN2A (D13211). Calcium phosphate was used to transiently transfect HEK cells in a 24-well plate with 500 ng of cDNA at a ratio of 1:1:5 (GluN1:GluN2A:GFP). Four hours after transfection, NMDAR antagonists D,L-2amino-5-phosphonovalerate (200 μM, DL-APV) and 7-chlorokynurenic acid (200 μM) were added to the culture medium to decrease the cytotoxic effect of NMDAR expression.

### Example 3: Stably Expressing HEK Cells

cDNAs for mouse Gabra1, Gabrb2, and the long form of Gabrg2 were subcloned into the pAC156 plasmid, a generous gift from Albert Cheng. The cDNAs were driven by an EF1alpha promoter. A PGK promoter-driven puromycin resistance cassette was also present in pAC156; both cassettes were flanked by piggybac transposon arms. All three plasmids were co-transfected with the mPB piggybac transposase into HEK 293 cells, selected by puromycin, and sorted into single cells. Clones were assayed for Gabra1, Gabrb2, and Gabrg2 expression by immunofluorescence, and one clone was expanded for further study and use in this description herein. Trypsin was used to dissociate the cells and plated on the same coverslips, as mentioned above, 24-72 hours before experimentation (shorter time and less PDL for lifted cell, and the inverse for excised patches).

### Example 4: Primary Neuronal Culture

Primary cortical neurons were cultured from the E17.5 rat embryos of either sex. Briefly, cortices were dissected from the embryos and trypsinized (0.25%, 37° C.). After rinsing cortical particles with warm Hank's balanced salt solution (HBSS, containing 10 mM HEPES, 2 rinsed), the particles were homogenized in minimum essential media (MEM, Cellgro) containing 10% FBS (MEM/FBS). Cells were plated on coverslips coated with 0.5-1 mg/ml poly-L-lysine in MEM/FBS. Two hours after plating, media was removed and replaced with glia-conditioned Neurobasal medium (Life Technologies, incubated on secondary mouse glia for 24 hrs) with 1× Glutamax (Life Technologies) and 1×B-27 (Life Technologies). Neurons were cultured at 5% CO₂ and 37° C., and every 3-4 days, a 50% media replacement was performed. Neurons were used at DIV 14-21. These procedures were approved by the Emory University Institutional Animal Care and Use Committee, and they were performed in accordance with state and federal Animal Welfare Acts and the policies of the Public Health Service.

### Example 5: Whole-Cell Voltage-Clamp Recordings

Whole-cell voltage-clamp recordings were performed with thin-walled borosilicate glass electrodes (3-6 MΩ, TW150E-4, World Precision Instruments) filled with solution containing (in mM) 110 Cs-gluconate, 30 CsCl, 5 HEPES, 4 NaCl, 0.5 CaCl₂, 2 MgCl₂, 5 BAPTA, 2 NaATP, 0.3 NaGTP (pH 7.35). The extracellular recording solution contained (in mM) 150 NaCl, 10 HEPES, 3 KCl, 0.5 CaCl₂, 1 MgCl₂, and 0.01 EDTA (pH 7.4). Whole cell recording from primary cortical neurons (examples showing alternative experiment paradigm in the supplemental) were obtained utilizing an internal solution, containing (in mM) 115 K-gluconate, 20 KCl, 10 HEPES, 2 Mg₂ATP, 0.3 NaGTP, and 10 Na₂Phosphocreatine (pH 7.35), and external solution stated above but with 1 mM CaCl₂. The electrode cleaning solution (2% Tergazyme in water) was made fresh daily. Cleaned electrodes were washed in appropriate internal solution. All solutions were filtered (0.45 μm or 0.22 μm). Responses were recorded using a Multiclamp 700B (Molecular Devices), filtered at 10 kHz (−3 dB), and digitized at 20 kHz.

### Example 6: Analysis and Statistics

Whole-cell rapid solution exchange experiments were analyzed using custom algorithms (Matlab, Mathworks). The desensitization and deactivation time courses were fitted by exponential functions based on receptor type. For NMDAR desensitization and GABA_{A}R deactivation, they were fit by one exponential function,

$$I = A * {e}^{\frac{- time}{τ}} + C$$

where I was the current response, A was the amplitude of the response, time is time, r is the time constant, and C is an offset constant. NMDAR deactivation and GABA_{A}R desensitization was fit with a dual exponential function,

$$I = {A}_{f} * {e}^{\frac{- time}{{τ}_{f}}} + {A}_{s} * {e}^{\frac{- time}{{τ}_{s}}} + C$$

the two exponentials are designated as fast (A_{f}, τ_{f}) and slow (Aₛ, τₛ). For dual exponential fits, a weighed tau (τ_{w}) was calculated,

$${τ}_{w} = \frac{{A}_{f} * {τ}_{f} + {A}_{s} * {τ}_{s}}{{A}_{f} + {A}_{s}} .$$

The Fisher's exact test, two tailed, was used where noted. Mean±SEM (standard error of the mean) is used unless otherwise noted.

### Example 6: Results

For efficient traditional pharmacological experimentation, one must ensure the viability of the available cell pool during sequential experimentation. Especially for ligand-gated ionotropic receptors, this is achieved by lifting cells or pulling patches from the coverslip and performing solution application far from the cells remaining on the coverslip (FIG. 2B). This procedure can be more straightforward than translating the manifold to the cell locations. It was first set out to ensure that this could be done reproducibly by the robotic system since achieving accurate placement of all components is essential for efficient data collection with minimal operator effort. The patcherBot_{Pharma} was verified for traversal of the recording electrode distances on the millimeter scale while ensuring micrometer scale precision at the interface of a multi-barrel flow pipe, given that piezoelectric translators typically have maximum ranges from 100-300 μm. This is especially important since in one complete cycle of the patcherBot_{Pharma} operation (patching, experiment manipulation, and electrode cleaning) the electrode will translate roughly 150 mm.

The patcherBot_{Pharma} needs to achieve this high level of accuracy and precision at the solution manifold without necessitating manual, time-intensive error correction. Typically, the placement of the electrode at the solution interface is established visually at a predesignated location (beginning of the recording session) then test pulses are conducted to ensure proper placement taking at least 30 second for a highly skilled operator. To test the ability to return to the critical location, the electrode was translated through the various positions required to patch sequentially (4×). After each cycle, the solution exchange around an open-tip electrode was measured by triggering a piezoelectric translation of the solution manifold (exchanging extracellular buffer and a partial salt solution containing 50% extracellular buffer and 50% H₂O). The electrode could be repeatability positioned while retaining the fast solution exchange time, and without placement errors that can lead to recording artifacts (i.e., straying into the adjacent lane before the jump, FIG. 2C).

Lifting cells in the whole-cell configuration and pulling outside-out patches are two of the most common methods of studying ligand-gated ionotropic receptors using rapid solution exchange manifolds. For lifting cells in the whole-cell conformation, as shown in FIG. 2D, a segmented (100 step) spiral translation method, represented by FIG. 2E, was implemented while applying a light suction on the pipette (−40 mbar). In applying this method, the cells were reliable lifted while retaining the high-resistance seal that was obtained while breaking through (FIG. 2F). For pulling outside-out patches, as pictured in FIG. 2G, a segmented (100 step) arc translation method, represented by FIG. 2H, was implemented while the pipette was at atmospheric pressure (0 mbar). In applying this method, outside-out patches were repeatedly pulled and/or lifted, achieving the characteristic low capacitance and high resistance of this patch-clamp conformation (FIG. 2I).

With these new functionalities, this system proved capable of performing rapid solution exchange experiments as well as executing precise solution application. To demonstrate these capabilities, receptor responses were recorded from two synaptic ligand-gated ionotropic receptors, GABA_{A}R and NMDARs, using the patcherBot_{Pharma} (FIGS. 3A and 3B). As expected, the patcherBot_{Pharma} was capable of recording NMDAR responses from transiently transfected HEK cells that were lifted off the bottom as well as from outside-out patches excised from HEK cells (FIG. 3A). Additionally, the patcherBot_{Pharma} was capable of recording GABA A R responses from stably expressing cells, including both long agonist applications as well as brief agonist applications (5 ms, FIG. 3B). In addition to this experimental protocol, the patcherBot_{Pharma} is programmed to conduct many other commonly used solution exchange protocols (FIGS. 6A through 6F) as well as voltage-clamp and current-clamp protocols. These can be employed to measure neuronal activity or study specific voltage-gated channels expressed in heterologous cells. The patcherBot_{Pharma} can implement these experimental protocols on adherent cells, lifted cells or patches pulled from cells, paired with solution control to measure channel responses in different conditions (FIGS. 7A through 7G).

A series of pharmacology experiments were performed on GABA_{A}R and NMDARs where rapid agonist application was applied to excised outside-out patches to assess patcherBot_{Pharma} performance on the minimum processes required in an experiment, described in more detail in Table 1). Assessment of the overall performance of the patcherBot_{Pharma} for both glutamate and GABA receptors revealed that a giga-ohm resistance patch (gigaseal patch) was obtained 81.2% of the time (108 of 133 attempts). After a gigaseal was achieved, successful break-in occurred 96.3% of the time to establish the whole-cell conformation (104 of 108 gigaseals). After whole-cell configuration stabilization, the success rate of excising an outside-out patch was 76.0% (79 of 104 whole-cell conformations). The successful completion of an experiment based on every outside-out patch pulled was 74.7% (59 of 79 outside-out patches). Subsequent failure to complete an experiment after obtaining an outside-out patch was due either to the lack of detectable receptor response upon agonist application or patch integrity breakdown after initiating the experimental recordings. Taken together the overall success of the patcherBot_{Pharma} was 44.4% (59 of 133 attempts). In examining the nature of failed experiments, the yield of the system is largely based on two main factors, electrode placement and biological factors.

TABLE 1
Success rates of steps in the patcherBotPharma process.

| | | | | | | | Overall |
|---|---|---|---|---|---|---|---|
| | | | | O-O | Successful | Zero | Exp. |
| | Attempts | GS | WC | Patch | Exp. | Current | Success |
| All GABA_{A}R and | | | | | | | |
| NMDAR | 133 | 108 | 104 | 79 | 59 | 19 | |
| NMDAR (transient) | 82 | 64 | 60 | 42 | 28 | 14 | |
| GABA_{A}R (stable) | 51 | 44 | 44 | 37 | 31 | 5 | |
| (computer vision) | 15 | 9 | 9 | 6 | 6 | 0 | |
| (operator assisted) | 36 | 35 | 35 | 31 | 25 | 5 | |
| All GABA_{A}R and NMDAR | | 81.2% | 96.3% | 76.0% | 74.7% | 24.1% | 44.4% |
| NMDAR (transient) | | 78.0% | 93.8% | 70.0% | 66.7% | 33.3% | 34.1% |
| GABA_{A}R (stable) | | 86.3% | 100.0% | 84.1% | 83.8% | 13.5% | 60.8% |
| (computer vision) | | 60.0% | 100.0% | 66.7% | 100.0% | 0.0% | 40.0% |
| (operator assisted) | | 97.2% | 100.0% | 88.6% | 80.6% | 16.1% | 69.4% |

One major contributing biological factor to experiment failure was the efficiency in the transient cDNA transfection process used to express the NMDARs. Overall, there was a higher success rate in achieving a high-quality recording from the stably expressing GABA_{A}R cells (31 successes out of 51 total attempts) than the transiently transfected NMDAR cells (28 successes out of 82 total attempts, Fisher's exact test, p=0.0039). Despite expression of GFP, which was coexpressed with NMDAR subunits, 14 of the 42 pulled patches did not have a current response of a sufficient amplitude. By contrast, the GABA A R cell line had a trend of higher reliability: only 5 of the 31 outside-out patches failed to have detectable current. This suggests that enhanced yield could result from improved molecular biology methods. Outside of those biological inefficiencies, monitoring the operation of the patcherBot_{Pharma} suggests that the failures at the gigaseal formation step and the outside-out patch-pulling step are due to slight errors (1-3 μm) in optimally placing the electrode. In this dataset, a subset of experiments were performed where an experimenter manually intervened by controlling the final placement of the electrode once the patcherBot_{Pharma} had positioned the electrode 100 μm above the next selected cell. In these operator-assisted experiments, the gigaseal yield was higher with 97.2% and the patch-pulling yield was 88.6%. Specifically, in obtaining gigaseals, the operator-assisted trials resulted in 35 successes from 36 attempts compared to 9 successes from 15 attempts (Fisher's exact test, p=0.0016). Additionally, in excising outside-out patches, the operator-assisted trials resulted in 31 successes from 35 attempts compared to 6 successes from 9 attempts (Fisher's exact test, p=0.1383). The overall yield (successful experiment compared to attempt) of these operator-assisted runs was 69.4% (25 good experiments of 36 attempts), as compared to the −40% success rate of the other experiments (6 good experiments of 15 attempts, Fisher's exact test, p=0.0645). Fully automated electrode placement implemented in the patcherBot_{Pharma} relies on machine vision using camera pixel intensity cross-correlation methods to align a previously stored image of the cell and electrode to make corrections at the beginning of each attempt. These methods work well in placing the electrode somewhere on a cell (˜10 μm precision) without operator intervention but lack the accuracy to place it optimally (<1 μm), which appears to have a large impact on overall success. In addition to the losses in efficiency, the machine vision processes are slow due to the necessity to move the electrode or microscope to check for positioning errors. The process time during fully automated patcherBot_{Pharma} operation takes on average 267±35 s (mean±Standard Deviation, SD) to correct the manipulators, land the electrode on the cell and break-in to the whole-cell conformation. This is compared to 74±10 s (mean±SD) for the operator-assisted patcherBot_{Pharma}, where robotic translations move the stage to the next cell and places the electrode just above the cell (100 μm) before the operator places the electrode on the cell and, in this case also, forms a gigaseal followed by the automated break-in process. Thus, the patcherBot_{Pharma} can operate fully autonomously but the speed and performance can be improved by operator intervention during key steps with the current techniques of position error correction.

Operating in this manner, with minor manual interaction, the patcherBot_{Pharma} can collect experiment electrophysiology recordings proficiently, which is exemplified by a representative run of the patcherBot_{Pharma} from the results mentioned previously (FIG. 4). In this experimental run, the patcherBot_{Pharma} was programmed to collect four-phase recordings. During each phase, five technical replicate sweeps were collected, a 10 sec sweep with agonist applied for 0.5 sec. Following each set of recordings, the patch was blown off with high pressure and the open-tip exchange time was determined to validate the electrode positioning. On average, the recording time and position validation totaled 11.2 min. If the patcherBot_{Pharma} detects inadequate patch formation, after the outside-out patch procedure, it terminates the recording and moves on to the next cell, spending only 1.4 min in doing so. Over this 3.8 hour recording session, 15 cells were attempted to be patched, yielding 12 successful recording sets. During this time, the operator only interacted with the patcherBot_{Pharma} for 15.5 min during recording (7.1% of the experimental run time) after the 10.3 min of calibration and cell selection. The patcherBot_{Pharma} was recording data for 2.6 hours, which amounts to 72.0% of the operation time. The GABA_{A}R responses that were collected were of high quality, shown in FIG. 4, and similar to those previously reported (Table 2). Additionally, the placement of the electrode resulted in consistent solution exchange times after each patch recording, as Illustrated in FIG. 4.

TABLE 2
Summary of activation and deactivation parameters of GABA_{A}R and
NMDARs from FIGS. 3A, 3B, and 4.
Rapid application of 1 mM GABA or Glutamate for 1 sec

| | | | Response | Response | | | | |
|---|---|---|---|---|---|---|---|---|
| | | Patch | Peak | Steady State | Desensitization | | | |
| | | Leak | Amplitude | Amplitude | Extent | Desensitization | Deactivation | |
| | | (pA) | (pA) | (pA) | (SS/Peak %) | Tau (ms) | Tau (ms) | N |
| GABA_{A}R | −40 mV | 24.9 ± 18.7 | 586 ± 150 | 170 ± 54 | 26.4 ± 1.8% | 681 ± 130 | 216 ± 29 | 12 |
| | −60 mV | 48.3 ± 36.1 | 173 ± 48 | 35.9 ± 13.6 | 24.8 ± 1.1% | 965 ± 298 | 133 ± 6 | 12 |
| NMDAR | −40 mV | 38.6 ± 32.1 | 271 ± 104 | 130 ± 68 | 34.2 ± 6.4% | 388 ± 75 | 71.9 ± 11.0 | 11 |
| GlnN2A WT | −60 mV | 140 ± 42.6 | 326 ± 180 | 215 ± 139 | 54.8 ± 6.9% | 984 ± 192 | 81.3 ± 18.4 | 7 |

Data shown represents the average ± SEM.

A case study measuring the main actions of a widely used anesthetic, propofol (PRO), was performed to highlight the operational procedure and capability of the patcherBot_{Pharma} in performing an extended and tedious patch-clamp electrophysiology experiment. Propofol's main clinical actions are produced by prolonging the deactivation of GABA_{A}R and have been well characterized. The patcherBot_{Pharma} was run with operator assistance for initial electrode placement, as illustrated in FIG. 5A, followed by automatic or partially manual patch formation to optimize the time of biological data collection by the patcherBot_{Pharma}, as illustrated in FIGS. 5B and 5C. An 8-point concentration response curve was collected of propofol's effects on GABA_{A}R deactivation and split it into two sets with a propofol-free control before and after drug application, as illustrated in FIG. 5D. In four, half-day recording sessions (2 per each concentration set) totaling 12.95 hours of patcherBot_{Pharma} operation, 42 recordings were attempted with 39 obtaining gigaseal patches, 28 achieving whole cell conformations, 24 successful pulled outside-out patches, and 18 completed experiments (including 6 incomplete) that yielded 113 data points (FIGS. 5E and 5F, Table 3, Table 4). After eliminating the recordings with too large a leak current, too small a response amplitude, or recording artifacts, 71 data points were left that were used to calculate the concentration-response relationship of propofol's ability to prolong the deactivation of GABA_{A}Rs (EC₅₀=11.8±4.6 μM, FIGS. 5E and 5F).

TABLE 3
Census of robotic operation and operator interaction time for the propofol case study.

| | Total | Electrode Cleaning | Data Collection | Patch Establishment | | Non-Recording |
|---|---|---|---|---|---|---|
| | Operation | (Robotic Control) | (Robotic Control) | (Operator Control) | | Time [symbol] |
| Total Time | 12.95 hrs | 1.39 hrs | 9.07 hrs | 2.49 hrs | | 3.88 hrs |
| (Percent) | | (10.7%) | (70.0%) | (19.3%) | | (30%) |
| Time Per Cycle | | 1.99 mins | 24.6 mins | 3.55 | mins | 5.54 mins |
| (successful cycle) | | | | | | |
| Time Per Data Point | | | 4.1 mins [symbol] | 2.1 | mins [symbol] | |
| (successful cycle) | | | (~1 min solution change time) | | | |
| | | | (1.67 min data collection) | | | |

^{†} The non-recording time is the time the robot is not performing the data collection protocol.
^{‡} This rate represents the total time the operator spent interacting with the patcherBot_{Pharma} during the entire experiment performance (cell selection, solution maintenance, electrode placement on the cell, and gigaseal formation). The theoretical maximal efficiency of data collection per the operator's effort would be 0.97 mins of the operator's time per data point. [symbol]
Each data collection phase equals the solution change time plus the data collection time, however the mean time per data point reflects the additional time needed to pull the patch and validate the jump at the end of the experiment averaged into the timing for each phase. [symbol]
indicates data missing or illegible when filed

TABLE 4
Concentration response of propofol (PRO) on GABA_{A}R activation
and deactivation.

| | | | | | | |
|---|---|---|---|---|---|---|
| | Patch Leak | Response Peak | Response Steady State | Desensitization | Deactivation | |
| GAB_{A}R | (pA) | Amplitude (pA) | Amplitude (pA) | Extent (SS/Peak %) | Tau (ms) | N |

Rapid application of 1 mM GABA for 0.5 sec—Propofol concentration set 1

| | | | | | | |
|---|---|---|---|---|---|---|
| GABA | 20.9 ± 7.3 | 133 ± 36 | 51.1 ± 12.3 | 39.3 ± 2.0% | 129 ± 11 | 8 |
| Control | | | | | | |
| 0.5 μM PRO | 28.9 ± 13.5 | 100 ± 20 | 40.4 ± 8.9 | 39.6 ± 1.3% | 116 ± 13 | 8 |
| 2 μM PRO | 16.7 ± 4.5 | 109 ± 32 | 45.6 ± 14.9 | 40.0 ± 1.7% | 128 ± 13 | 6 |
| 10 μM PRO | 18.8 ± 4.8 | 96.0 ± 25.2 | 42.1 ± 11.6 | 43.1 ± 2.2% | 208 ± 20 | 6 |
| 50 μM PRO | 16.0 ± 3.0 | 63.9 ± 17.8 | 23.5 ± 7.4 | 33.8 ± 2.7% | 331 ± 21 | 7 |
| PRO | 15.9 ± 2.8 | 66.7 ± 22.6 | 29.2 ± 11.3 | 41.8 ± 1.7% | 143 ± 25 | 6 |
| washout | | | | | | |

Rapid application of 1 mM GABA for 0.5 sec—Propofol concentration set 2

| | | | | | | |
|---|---|---|---|---|---|---|
| GABA | 30.6 ± 6.2 | 93.6 ± 6.8 | 30.5 ± 8.1 | 31.8 ± 7.7% | 130 ± 27 | 6 |
| Control | | | | | | |
| 1 μM PRO | 36.5 ± 6.4 | 72.2 ± 18.0 | 24.2 ± 6.6 | 34.9 ± 6.7% | 110 ± 15 | 6 |
| 5 μM PRO | 34.3 ± 8.2 | 37.8 ± 9.1 | 11.1 ± 2.4 | 30.0 ± 5.1% | 198 ± 29 | 5 |
| 20 μM PRO | 42.1 ± 8.0 | 21.5 ± 4.6 | 6.1 ± 2.2 | 25.2 ± 6.6% | 328 ± 53 | 5 |
| 100 μM PRO | 29.0 ± 5.9 | 8.9 ± 1.2 | 5.8 ± 7.5 | — | 415 ± 226 | 5 |
| PRO | 24.8 ± 10.1 | 20.1 ± 6.3 | 5.3 ± 3.4 | 25.2 ± 13.5% | 129 ± 12 | 3 |
| washout | | | | | | |

Data shown represents the average ± SEM.

Of the 12.95 hours of recording, the operator interacted with the patcherBot_{Pharma} for 2.49 hours and the patcherBot_{Pharma} collected experimental recordings for 9.07 hours, with an additional 1.39 hours of other automated processing (Table 3). The 2.49 hours of operator interaction includes cell selection, solution maintenance, electrode placement on the cell, and gigaseal formation. In each iteration of the patcherBot_{Pharma} process, it spent 1.99 minutes cleaning the electrode and the operator spent ˜2-3 minutes placing the electrode on the cell and establishing the whole-cell conformation. If everything was successful, the patcherBot_{Pharma} would then proceed to collect the experimental data—in total a 24.6 minute process. If there was an issue with the stability of the patch during the process of pulling the outside-out patch (1.73 min process), the patcherBot_{Pharma} would clean the electrode and be ready for the next attempt in less than 2 minutes. Although, the experiment yield was not overly high (24/42 attempts were successful) this did not hinder the performance of the patcherBot_{Pharma} greatly (Table 5). If every patch attempt was successful, the theoretical maximum number of experiments the patcherBot_{Pharma} could have performed in 12.95 hours was 25.8, which is only modestly higher than the 18 that were successfully performed manually (70% full experiments performed divided by the maximum). Moreover, the rate of data collection, in terms of operator effort, of the patcherBot_{Pharma} process time was 2.1 minutes per data point. Should patching efficiency be improved further, the theoretical minimum of operator effort can be reduced to 0.97 minutes per data point.

TABLE 5
Performance of the patcherBot_{Pharma} in the propofol case study.

| | | | Theoretical |
|---|---|---|---|
| | Counts | Yield | Max^{†} |
| Patch Attempts | 42 | | |
| Successfully established gigaseals | 39 | 93% | |
| Whole cell conformations obtained | 28 | 72% | |
| Outside-out patches obtained | 24 | 86% | |
| Experiments started | 24 | — | |
| Successful Experiments | 18 | 75% | 25.8 |
| Total Data Points Collected | 113 | | 154.8 |
| Data Points Passed Quality Control | 71 | | |

^{†}The theoretic maximum values were determined by taking the total operation time divided by the total time for one successful cycle. Since there are 6 collected data points per experiment, the theoretical maximum for total collected data points equals the number of experiments multiplied by a factor of 6.

### Example 7: Discussion

Patch-clamp electrophysiology research is a powerful technique, yet many scientists are dissuaded from learning and utilizing this approach because of its time-consuming nature, in terms of both training and execution. Even for skilled practitioners, the complexity and effort required for comprehensive pharmacology experiments (pharmacological screening or evaluation of full concentration-response relationships) can be impractical. Here, the capabilities of the patcherBot_{Pharma} have been demonstrated for ligand gated ionotropic receptor pharmacological screening, which makes patch-clamp electrophysiology experimentation rapid, less skill intensive, and more reliable. The automation of the patcherBot_{Pharma}, namely precise and accurate electrode translations, solution handling, electrode cleaning and rapid solution exchange, greatly expands the repertoire of experiments that the patcherBot and other automated systems can perform. This allows one to conduct nearly any pharmacological experiment typically performed on ligand-gated or voltage-gated ion channels using the patcherBot_{Pharma} (e.g. FIGS. 6A through 7G). Additionally, the patcherBot_{Pharma} has the flexibility to be retooled as needed based off a traditional patch-clamp rig and can run autonomously or with minimal operator intervention to suit the experimental situation. Thus, the patcherBot_{Pharma} could be set up to patch adherent cells and applied compounds via the bath input, if desired, and the fully automated capabilities of the system will be retained if all test compounds can be fully washed out.

The patcherBot_{Pharma} has very high yield (80-100%) of obtaining giga-ohm resistance patches and of breaking-in to achieve the whole-cell patch conformation. The methods employed to lift isolated patch-clamped cells and to pull outside-out patches are highly reliable (70-90% yield). These capabilities allow the patcherBot_{Pharma} to spend more time performing the intended electrophysiology experiment and less time in the process of manually guiding the position of the patch electrode throughout the course of the full experiment. With this improved system, the primary determinants for whether a particular experimental attempt concludes in a successful recording, relies more on biological factors than robotic or operator factors. In the experiments with heterologous expression systems (namely transfected HEK cells), the yield in high quality recordings, with high receptor expression, of the patcherBot_{Pharma} reaches 60-70% of the cells attempted. With this high efficiency of data collection, the system could rapidly replicate previous datasets by reducing the time it takes to produce an 8-point concentration response curve of the effects of propofol on GABA_{A}R deactivation from weeks/months of recording down to −13 hours of recording.

This system retains the full capabilities that traditional electrophysiology rigs have. Solution exchange times were observed with the larger three-barreled manifold in the low millisecond range (−1-2 ms), which could be reduced further (<1 ms) using different solution manifolds. This allows for accurate experimentation and can be used to study rapidly desensitizing receptors, which cannot be measured on commercially available multi-well high-throughput patch clamp instrumentation. The patcherBot_{Pharma} system largely comprises typical components of a conventional electrophysiology rig, and thus does not require a substantial or prohibitive cost to upgrade. Running costs are low, comparable to the cost of operating a traditional patch-clamp rig, and primarily include the cost of the preparation (cell culture costs) and compounds being evaluated. There are no additional changes in running costs based on each data point collected, except for reduced glass consumption and perhaps reduced preparation costs that come with more efficient recording. However, as the patcherBot_{Pharma} can be in operation for extended periods of time and can execute experiments at a high rate, the running costs based on each day of operation may be, in fact, higher due to the increased bath solution usage and increased use of pharmacological compounds.

There are several improvements to the patcherBot_{Pharma} that could further increase its abilities and productivity. Enhanced machine vision correction methods could allow for more precise placement of the electrode with less computation time, thus increasing the unattended success rate and reducing human effort. Algorithms for cell detection could be employed to make cell selection agnostic, with further reduction in human effort and bias. Systematic collection of data will aid in meta-analysis of experiments, which could identify unrecognized factors that influence experimental results or experimental variability.

The patcherBot_{Pharma} facilitates pharmacological experimentation on ligand gated channels through increased productivity and the ability to address labor-intensive questions (collecting multiple concentration data points or testing more constructs). This allows more complex experimental designs that include higher replicates and more controls. Many neuroscience studies have been cited as having low power in their experimental design, which could be rectified by utilizing the patcherBot_{Pharma} Additionally, the patcherBot_{Pharma} reduces the chance of human bias when collecting data, as the experiment protocols are explicitly defined prior to experiment execution. Moreover, methods to introduce blinding in the experimental design could be employed along with automated analysis to allow one to easily jump to the final analyzed data point after conducting the experiment. The data collected by the patcherBot_{Pharma} might be more reproducible due to enhanced transparency, as the full patcherBot_{Pharma} experiment data log could be documented along with the results. With the reduction in human effort that comes with operating the patcherBot_{Pharma}, it becomes feasible that a single person could operate multiple patcherBot_{Pharma} at once for increased data collection. In summary, the patcherBot_{Pharma} enhances the capabilities of a researcher utilizing patch-clamp approaches by decreasing operator interaction time, reducing human bias, increasing experiment yield, allowing more complicated experimental designs, and enabling experiments that require high volumes of recordings.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A system configured to aid in a detection process, the system comprising:
a pipette tip manipulable between a sample area and an active agent exposure zone; and
a controller configured to:
form a high-resistance seal between the pipette tip and at least a portion of a biological sample target located in the sample area; and
manipulate the pipette tip between the sample area and the active agent exposure zone to isolate the sealed portion of the biological sample target from other biological sample targets contained in the sample area.

2. The system of claim 1, wherein the active agent exposure zone comprise a delivery port configured to distribute an active agent to the active agent exposure zone;
wherein the detection process is configured to detect an effect of the distributed active agent on the biological sample target.

3.-4. (canceled)

5. The system of claim 1, wherein manipulating the pipette tip comprises translocating the pipette tip and the sealed portion of the biological sample target from the sample area to the active agent exposure zone.

6. The system of claim 5, wherein the controller is configured to translocate the pipette tip from the sample area with at least one of a pre-determined pattern of motion, a pre-determined speed, or a pre-determined pressure.

7. The system of claim 6, wherein the pre-determined pattern of motion comprises one or more translations within an x-plane, a y-plane, a z-plane, or combinations thereof.

8. The system of claim 6, wherein the pre-determined pattern of motion comprises one or more of a spiral motion, a helical motion, a wave motion, a square motion, a triangle motion, a sawtooth motion, a reciprocating motion, an oscillating motion, a rotary motion, a linear motion, a translatory motion, a rectilinear motion, a curvilinear motion, a parabolic motion, a circulatory motion, a vibratory motion, a periodic motion, or combinations thereof.

9. The system of claim 6, wherein the controller is further configured to modify at least one of the pre-determined pattern of motion, the pre-determined speed, or the pre-determined pressure based on a change in the high-resistance seal.

10. A system configured to aid in a detection process, the system comprising:
an apertured surface manipulable between a sample area containing two or more samples and an active agent exposure zone; and
a controller configured to:
control the apertured surface into contact with at least a portion of a biological sample target of the two or more samples, wherein the detection process is configured to detect at least one characteristic of the biological sample target; and
isolate the contacted biological sample target from other of the two or more samples by manipulating the contacted portion of the biological sample target at the apertured surface from the sample area to the active agent exposure zone.

11. The system of claim 10, wherein a first characteristic of the biological sample target is a first electrical property of the contacted portion of the biological sample target at the apertured surface.

12. (canceled)

13. The system of claim 10, wherein the controller is further configured to construct a first temporal series of electrical property measurements indicative of initiating contact between the apertured surface and the biological sample target.

14. The system of claim 10, wherein the active agent exposure zone comprises a delivery port configured to distribute an active agent to the active agent exposure zone; and
wherein the controller is further configured to control the delivery port and the contacted portion of the biological sample target at the apertured surface within the active agent exposure zone to test for the detected at least one characteristic of the biological sample target before, during, and after exposure to the active agent from the delivery port.

15. The system of claim 10, wherein the active agent exposure zone comprises a delivery port configured to distribute an active agent to the active agent exposure zone; and
wherein the controller is further configured to control an amount of the active agent distributed from the delivery port.

16. (canceled)

17. The system of claim 11, wherein the contact between the apertured surface and the biological sample target comprises forming a high-resistance seal; and
wherein a second characteristic of the biological sample target is a second electrical property at the apertured surface indicative of the high-resistance seal.

18. (canceled)

19. The system of claim 13, wherein the controller is further configured to construct a second temporal series of electrical property measurements indicative of an effect of an active agent on the biological sample target.

20. The system of claim 17, wherein the controller is further configured to determine whether the apertured surface is losing contact with the biological sample target by detecting a change in the high-resistance seal.

21. The system of claim 10, wherein the contact between the apertured surface and the biological sample target comprises forming a high-resistance seal;
wherein the active agent exposure zone comprises a delivery port configured to distribute an active agent to the active agent exposure zone;
wherein a first characteristic of the biological sample target is a first electrical property indicative of initiating contact between the apertured surface and the biological sample target;
wherein a second characteristic of the biological sample target is a second electrical property at the apertured surface indicative of the high-resistance seal;
wherein the controller is further configured to construct a first temporal series of electrical property measurements to determine the first characteristic;
wherein the controller is further configured to construct a second temporal series of electrical property measurements indicative of an effect of the active agent on the biological sample target; and
wherein the first electrical property, second electrical property, first temporal series of electrical property measurements, and second temporal series of electrical property measurements comprises at least one of a current, a capacitance, a resistivity, a conductivity, a temperature coefficient of resistance, a dielectric strength, thermoelectricity, or a combination thereof.

22. The system of claim 1, wherein the controller is further configured to control the pipette tip between the sample area and a cleaning area;
wherein the cleaning area is configured to contain cleaning solution and fluidically separate the cleaning solution from the sample area.

23. The system of claim 1, wherein the controller is further configured to automate a whole-cell patch clamp recording of the biological sample target.

24. The system of claim 1, wherein the controller is further configured to automate an outside-out patch clamp recording of the biological sample target.

25.-26. (canceled)

27. The system of claim 1, wherein the detection process is one of:
achieving a whole-cell conformation such that the biological sample target at the pipette tip comprises an entire biological sample target; or
achieving a patch conformation such that the biological sample target at the pipette tip comprises a portion of the biological sample target.

28.-31. (canceled)

32. The system of claim 15, wherein the detection process comprises:
contacting the apertured surface and the biological sample target with a perfusion of the active agent within the active agent exposure zone; and
measuring the at least one characteristic.

33. The system of claim 32, wherein at least one characteristic of the biological sample target is selected from the group consisting of an electrical activity, a molecular activity, a drug screening property, biological sample target type, a biophysical property, and a genetic property.

34. (canceled)

35. The system of claim 10, wherein the detection process is a method for detecting an effect of an active agent on a cell;
wherein the biological sample target is a target cell;
wherein controlling the apertured surface and contacted biological sample comprises forming a high-resistance seal between the target cell and the apertured surface;
wherein isolating the target cell comprises translocating the apertured surface from the sample area in one or more translations within an x-plane, a y-plane, a z-plane, or combinations thereof; and
wherein the target cell is exposed to the active agent.

36. The system of claim 35, wherein the detection process is one of:
achieving a whole-cell conformation such that the target cell at the apertured surface comprises an entire cell; or
achieving a patch conformation such that the target cell at the apertured surface comprises a cell membrane patch.

37.-45. (canceled)
